(19) United States
(12) Patent Application Publication
(10) Pub. No.: US 2023/0310499 A1
(43) Pub. Date: Oct. 5, 2023

(54) PLASMA EXCHANGE REMOVAL OF SPD-L1

(71) Applicant: Mayo Foundation for Medical Education and Research, Rochester, MN (US)

(72) Inventors: Jacob J. Orme, Rochester, MN (US); Aaron S. Mansfield, Rochester, MN (US); Roxana S. Dronca, Rochester, MN (US); Haidong Dong, Rochester, MN (US); Jeffrey L. Winters, Rochester, MN (US)

(21) Appl. No.: 18/020,515

(22) PCT Filed: Aug. 12, 2021
(86) PCT No.: PCT/US2021/045687

§ 371 Date: Feb. 9, 2023

Related U.S. Application Data
(60) Provisional application No. 63/064,768, filed on Aug. 12, 2020.

(51) Int. Cl. A61K 35/16; A61K 39/395; A61P 35/00; G01N 33/574

(52) U.S. Cl. CPC A61K 35/16; A61K 39/3955; A61P 35/00; G01N 33/5743; G01N 33/57484; G01N 2333/70596

(57) ABSTRACT

Materials and methods for improving immunotherapy are provided herein. In some cases, the materials and methods can be used in the treatment of cancers.

### CROSS-REFERENCE TO RELATED APPLICATIONS

This application claims priority from U.S. Provisional Application Serial No. 63/064,768, filed Aug. 12, 2020.

### STATEMENT OF FEDERALLY SPONSORED RESEARCH

This invention was made with government support under CA197878 awarded by the National Institutes of Health. The government has certain rights in the invention.

### TECHNICAL FIELD

This document relates to materials and methods for removal of extracellular forms of PD-L1 (also referred to as B7H1) and/or extracellular vesicles (EVs) by therapeutic plasma exchange (TPE).

### BACKGROUND

Programmed cell death protein 1 (PD-1, CD279) is a cell surface receptor found on immune cells and other cells. Signaling through this receptor causes downregulation of the immune system. PD-1 signaling in immune cells is most commonly induced by programmed death-ligand 1 [PD-L1; also known as CD274 or B7 homolog 1 (B7H1)] on other cells. Many cancers have been shown to express ligand PD-L1 and signal immune cell downregulation through surface receptor PD-1, which can allow those cancers to escape anti-tumor immunity (see, e.g., Dong et al., Nature Medicine 5(12):1365-1369, 1999, https://doi.org/10.1038/70932; Dong et al., Nature Medicine 8(8):793, 2002, doi.org/10.1038/nm730; and Freeman et al., J Exp Med 192(7):1027-1034, 2000, doi.org/10.1084/jem.192.7.1027).

Inhibitors of PD-⅟PD-L1 interaction and/or other immune ligand/receptor interactions (commonly referred to as checkpoint inhibitors, immunotherapy, PD-(L)1 inhibitors, PD-1 inhibitors, PD-L1 inhibitors, or other similar terms) can abrogate the interaction of immunosuppressive ligands with their receptors, thus upregulating anti-tumor immunity. Checkpoint inhibitors have been approved for use in many different malignancies. For example, anti PD-1 antibodies such as pembrolizumab (MK-3475), nivolumab (BMS-936558), and pidilizumab can block ligands such as tumor-associated PD-L1 from interacting with PD-1 on tumor-reactive T cells, thus preventing tumor-induced T cell death. By blocking activation of this immune checkpoint, administered anti-PD-1 antibodies can improve a mammal’s immune responses against tumors. Likewise, anti PD-L1 antibodies such as durvalumab, avelumab, and atezolizumab can block the interaction of PD-L1 with PD-1 and can improve a mammal’s immune responses against tumors. However, response rates to checkpoint inhibitors are under 20% on average, and few cancers respond more than half of the time to PD-(L)1 inhibitors such as pembrolizumab or combination nivolumab and ipilimumab (Haslam and Prasad, JAMA Network Open, 2(5):e192535, 2019; Hellmann et al., New Engl J Med, 381(21):2020-2031, 2019; Rini et al., New Engl J Med, 380(12):1116-1127, 2019; and Robert et al., New Engl J Med, 372(26):2521-2532, 2015). In patients that do respond, tumors almost always eventually develop resistance (O′Donnell et al., Cancer Treatment Rev, 52:71-81, 2017).

A source of resistance to immunotherapies is extracellular PD-L1 and related substances. Trans-acting PD-L1 derives from malignant cells in three known forms: secreted splice variant PD-L1 (sPD-L1), ADAM10/ADAM17-shed sPD-L1, and evPD PD-L1-positive extracellular vesicles (evPD-L1) (see, e.g., Orme et al., Oncoimmunol, 9(1):e1744980, 2020; WO 2019/161129; Ando et al., Anticancer Res, 39(9):5195-5201, 2019; Fan et al., Ann Surg Oncol, 26(11):3745-3755, 2019; and Zhou et al., Cancer Immunol Res, 5(6):480-492, 2017). High levels of any of these trans-acting PD-L1 forms can predict poor prognosis and limited response to PD-(L)1 checkpoint inhibitors in cancer. In clinical trials, high levels of sPD-L1 and evPD-L1 predicted poor prognosis and response to immunotherapy (Ando et al., supra; Chen et al., supra; Costantini et al., OncoImmunol, e1452581, 2018; Dronca et al., Pigment Cell Melanoma Res, 28(6):768-769, 2015; Ha et al., Oncotarget, 7(47):76604-76612, 2016; Li et al., Clinical Breast Cancer, 19(5):326-332, 2019; Okuma et al., Lung Cancer (Amsterdam, Netherlands), 104:1-6, 2017; Okuma et al., Clinical Lung Cancer, 19(5):410-417.e1, 2018; Theodoraki et al., supra; Zhang et al., Thoracic Cancer, 6(4):534-538, 2015; and Zhao et al., Medicine, 96(7):e6102, 2017). Extracellular vesicles also have been implicated in disease processes such as aging, autoimmunity, heart disorders, infection, neurodegeneration, and obesity (Boulanger et al., 2017, Nature Rev Cardiol, 14(5):259-272, 2017; Huang-Doran et al., Trends Endocrinol Metabol, 28(1):3-18, 2017; Marcilla et al., J Extracellular Vesicles, 3(1):25040, 2014; Nakao et al., PLoS ONE, 6(10), 2011; and Thompson et al., Nature Rev Neurol, 12(6):346-357, 2016. No clinical intervention has previously been shown to mitigate these effects.

### SUMMARY

This document is based, at least in part, on the discovery that therapeutic plasma exchange (TPE) can be used a treatment to reduce circulating extracellular PD-L1 in patients with cancer. This document also is based, at least in part, on the development of methods for using TPE and immunotherapy in cancer patients identified as having levels of a substrate (e.g., a PD-L1 marker, such as sPD-L1 or evPD-L1) at or above a predetermined threshold. As described herein, for example, sPD-L1 levels above a threshold level (0.277 ng/mL) predicted inferior overall survival (OS) for patients with melanoma. In patients undergoing TPE for non-malignant indications, TPE sessions removed a mean 70.8% sPD-L1 and 73.1% evPD-L1 detectable in plasma. TPE also reduced total and ADAM10-positive EVs. Thus, the materials and methods described herein provide the first known clinical intervention to remove sPD-L1 and/or evPD-L1 from plasma in vivo, suggesting a role for TPE in cancer treatment along with immunotherapy. The methods described herein can reduce the ability of sPD-L1 to decrease the effectiveness of immunotherapy (e.g., inhibitors of PD-1/PD-L1 interaction) for treating cancer, and can increase the number of patients who may benefit from an anti-PD-1 antibody and/or anti-PD-L1 antibody treatment protocol. The methods and materials described herein therefore can provide improved responsiveness to immunotherapy, lengthened survival from cancer, and improved relief from symptoms. Such benefits can be experienced by cancer patients receiving one or more inhibitors of PD-1/PD-L1 interactions (e.g., an anti-PD-1 antibody and/or an anti-PD-L1 antibody), immunodeficient patients receiving one or more inhibitors of PD-1/D-L1 interactions (e.g., an anti-PD-1 antibody and/or an anti-PD-L1 antibody), and other patients receiving or planning to receive one or more inhibitors of PD-1/PD-L1 interactions (e.g., an anti-PD-1 antibody and/or an anti-PD-L1 antibody). In addition, the materials and methods provided herein also may be useful in patients with other EV-related conditions.

In a first aspect, this document features a method that includes (a) performing therapeutic plasma exchange (TPE) on a mammal identified as (i) having cancer and (ii) having a measured level of one or more markers in a biological sample that is equal to or higher than a threshold level, and subsequently (b) administering an immunotherapy to the mammal. The one or more markers can include soluble PD-L1 (sPD-L1). The threshold level can be from 0.27 ng/mL to 1.75 ng/mL sPDL1. The threshold level can be from 5 ng/mL to 10 ng/mL sPDL1. The one or more markers can include extracellular vesicle PD-L1 (evPD-L1). The one or more markers can include extracellular vesicles (EVs). The measuring can include performing an enzyme linked immunosorbent assay (ELISA). The measuring can include performing nanoflow cytometry. The immunotherapy can include an anti-PD-1 antibody or anti-PD-L1 antibody. The mammal can be a human. The cancer can be melanoma, renal cell carcinoma, mesothelioma, squamous cell cancer, a hematological cancer, neurological cancer, breast cancer, head and neck cancer, gastrointestinal cancer, liver cancer, pancreatic cancer, genitourinary cancer, bone cancer, bladder cancer, or vascular cancer.

In another aspect, this document features a method for treating a mammal identified as having cancer, where the method includes (a) measuring the level of one or more markers in a biological sample obtained from the mammal, (b) comparing the measured level of the one or more markers to a threshold level, (c) when the measured level is equal to or greater than the threshold level, performing TPE on the mammal, and subsequently (d) administering an immunotherapy to the mammal. The one or more markers can include sPD-L1. The threshold level can be from 0.27 ng/mL to 1.75 ng/mL sPDL1. The threshold level can be from 5 ng/mL to 10 ng/mL sPDL1. The one or more markers can include evPD-L1. The one or more markers can include EVs. The measuring can include performing an ELISA. The measuring can include performing nanoflow cytometry. The immunotherapy can include an anti-PD-1 or anti-PD-L1 antibody. The mammal can be a human. The cancer can be melanoma, renal cell carcinoma, mesothelioma, squamous cell cancer, a hematological cancer, neurological cancer, breast cancer, head and neck cancer, gastrointestinal cancer, liver cancer, pancreatic cancer, genitourinary cancer, bone cancer, bladder cancer, or vascular cancer.

In another aspect, this document features a method for treating a mammal identified as having a cancer that is resistant to immunotherapy. The method can include performing TPE on the mammal and administering an immunotherapy to the mammal. The mammal can have been identified as having a cancer resistant to immunotherapy by measuring the level of one or more immunosuppressive components in a biological sample obtained from said mammal, and determining that said level is equal to or higher than a given cutoff level. The one or more immunosuppressive components can include one or more of sPD-L1, evPD-L1, and evADAM10. The mammal can be a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C show that soluble PD-L1 (sPD-L1) suppresses antitumor immunity and predicts overall survival in patients with melanoma. FIG. 1A is a model showing three known tumor-derived extracellular PD-L1 forms - (1) evPD-L1, (2) ADAM10/ADAM17-cleaved sPD-L1 ectodomain, and (3) secreted splice variant sPD-L1 that can downregulate anti-tumor immunity and prevent response to PD-(L)1 inhibition.

FIG. 1B is a Kaplan-Meier plot showing significantly worse overall survival for patients with melanoma who exhibit high (≥ 0.277 ng/mL) versus low (< 0.277 ng/mL) plasma sPD-L1 levels (p=0.005). FIG. 1C is a graph plotting plasma mean sPD-L1 levels in patients with melanoma and healthy controls, showing that the melanoma patients exhibited a higher mean plasma sPD-L1 level (1.72 ng/ml) than the controls (0.773 ng/ml). *** P < 0.001.

FIGS. 2A-2C show that TPE significantly reduces plasma sPD-L1 levels. FIG. 2A is a model of the TPE procedure in which patient plasma is separated and replaced to extract noncellular substances confined to the plasma. FIG. 2B is a graph plotting plasma levels of sPD-L1 immediately prior to (Pre) and after (Post) TPE using albumin replacement fluid. TPE significantly reduced sPD-L1 levels in patient plasma by Wilcoxon signed-rank test (p<0.0001). FIG. 2C is a graph showing that in a typical timeline, patient sPD-L1 levels were reduced by each successive session of TPE (gray bars). See, also TABLE 5 and FIGS. 4 and 5.

FIG. 3 is a graph plotting baseline plasma sPD-L1 in normal controls versus patients undergoing TPE. Levels of PD-L1 in patients undergoing TPE were not significantly lower than those of matched normal controls. A statistical table including p value (two-sided Student’s t test), mean, and 95% confidence intervals is shown below the graph.

FIG. 4 is a series of graphs plotting plasma sPD-L1 in all TPE treatment courses (including sessions involving FFP) for 23 patients of the 25 patients studied. Treatment courses for each patient are shown. Dark gray bars represent TPE sessions in which FFP was given. Light gray bars represent TPE sessions in which no FFP (i.e., only albumin) replacement was given. The treatment course for Patient 22 is shown in FIG. 2C. Patient 6 was excluded for biotin use.

FIG. 5 is a graph plotting plasma sPD-L1 levels for all TPE treatment courses (including sessions requiring FFP). TPE significantly reduced sPD-L1 levels in all sessions, including those in which patients received donor FFP.

FIG. 6 is a graph plotting sPD-L1 levels in plasma from donors of FFP, showing variable levels of sPD-L1 by blood type.

FIGS. 7A-7D show that plasma exchange efficiently reduced total, PD-L1-positive, and ADAM10-positive extracellular vesicle (EV) levels in vivo. Plasma levels of total extracellular vesicles (EVs) immediately prior to (Pre) and after (Post) TPE are plotted. TPE significantly reduced total EVs (FIG. 7A, p<0.0001), PD-L1-positive EVs (FIG. 7B, p=0.028), and ADAM10-positive EVs (FIG. 7C, p<0.0001), but not CD61-positive EVs (FIG. 7D, p=0.94) by Wilcoxon signed-rank test. See, also, FIGS. 8-10 and TABLE 6.

FIG. 8 is a series of graphs plotting plasma EVs in all TPE treatment courses (including sessions requiring FFP) for 25 patients. For each patient, total plasma EV levels (top left), PD-L1-positive EV levels (top right), ADAM10-positive EV levels (bottom left), and CD61-positive EV levels (bottom right) over the course of TPE treatment are shown. Bars indicate TPE sessions, usually lasting 2 hours. Light-colored bars indicate that no FFP was received during the TPE session, while dark bars indicate that FFP was received during the TPE session.

FIG. 9 shows representative examples of plasma EV nanoflow plots. Pre-TPE (top row) and post-TPE (bottom row) nanoflow cytometry detecting total EVs, evCD61, evPD-L1, and evADAM10 for Patient 10 are shown.

FIG. 10 is a graph plotting total plasma EV per microliter levels in all TPE treatment courses, including those sessions in which FFP was received.

FIGS. 11A-11D are a series of graphs showing that FFP donor EV concentrations do not correlate with blood type. FIG. 11A, total EVs; FIG. 11B, PD-L1-positive EVs; FIG. 11C, ADAM10-positive EVs; and FIG. 11D, CD61-positive EVs.

FIG. 12 is a graph plotting plasma sPD-L1 levels in two melanoma patients who were treated with immunotherapy and still experienced disease progression. At progression, the patients had elevated sPD-L1. After undergoing TPE, the levels were dramatically reduced. These patients have not had further progression of their disease.

FIG. 13 is a diagram depicting a multistep process for detecting one or more substrates (200) through such means as ELISA, nanoflow, or another mechanism; selection of TPE treatment (210) for patients with substrates over a given cutoff in one or more sessions for the purpose of removing the one or more substrates; and administration of immunotherapy such as pembrolizumab, atezolizumab, ipilimumab, and/or another immunotherapy (220).

### DETAILED DESCRIPTION

This document provides materials and methods for treating mammals (e.g., human patients) that have cancer and are resistant to immunotherapy. For example, the methods provided herein can be used to treat cancer patients who are “PD-1 resistant” (also referred to as “anti-PD-1 antibody resistant” and “anti-PD-1 non-responder”) in that they do not respond, or have a reduced response, to treatments targeted to PD-1 (e.g., anti-PD-1 antibodies) due to, for example, interference from sPD-L1. The methods provided herein also can be used to treat cancer patients who are “PD-L1 resistant” (also referred to as “anti-PD-L1 antibody resistant” and “anti-PD-L1 non-responder”) in that they do not respond, or have a reduced response, to treatments targeted to PD-L1 (e.g., anti-PD-L1 antibodies) due to, for example, interference from sPD-L1.

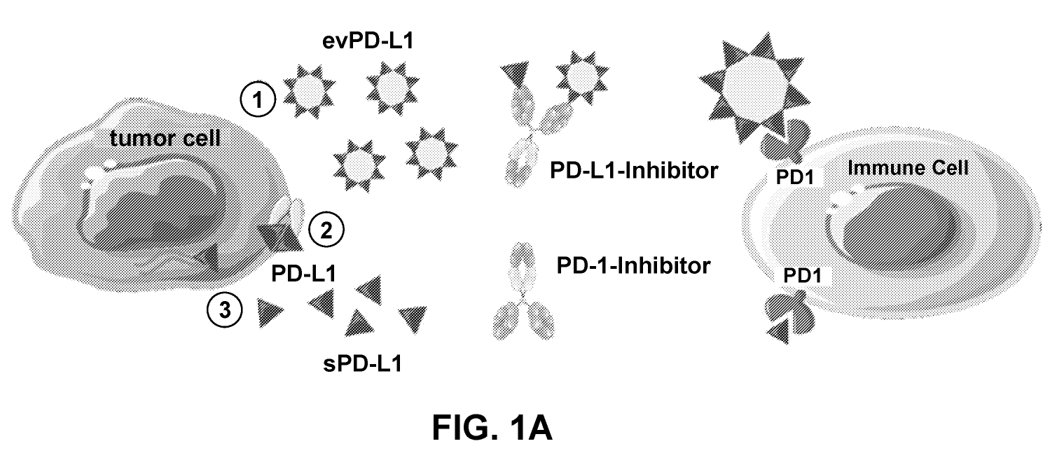

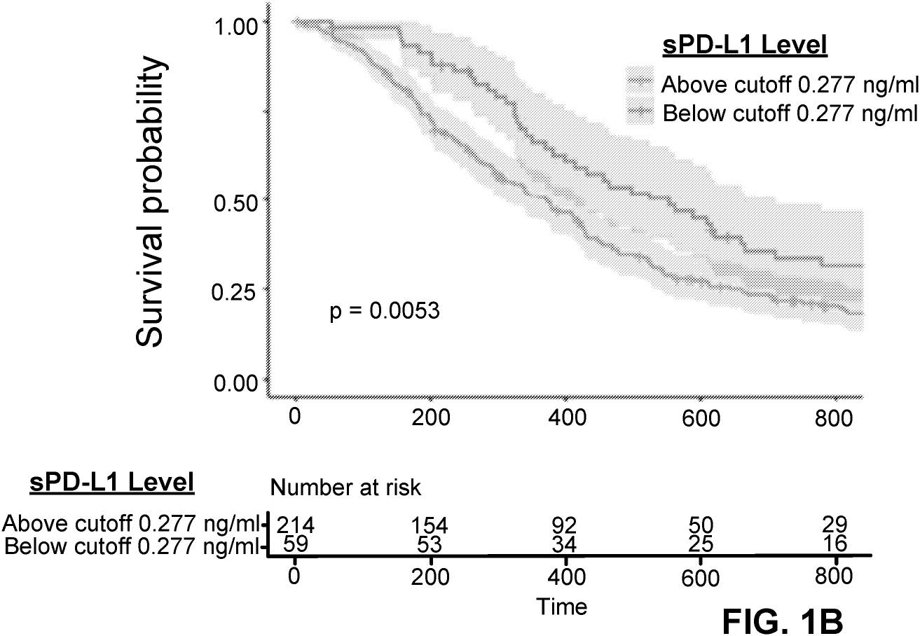

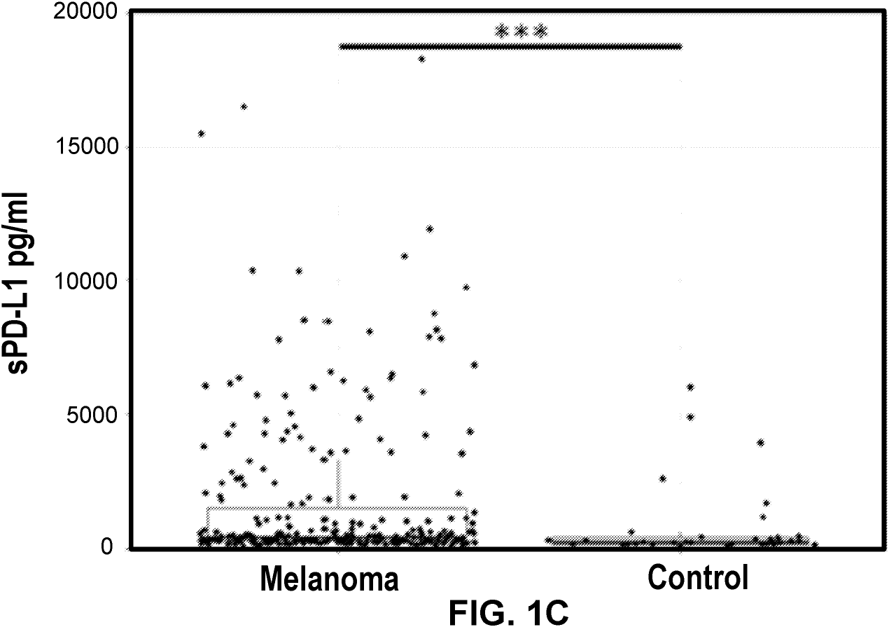

As noted above, increased levels of extracellular PD-L1 (e.g., sPD-L1 and evPD-L1) can reduce the effectiveness of immunotherapy. FIG. 1A is a diagram depicting how extracellular PD-L1 can cause significant downregulation of immunity in different forms. Tumors and other cells can produce (1) evPD-L1, (2) ADAM10/ADAM17-cleaved sPD-L1 ectodomain, and (3) secreted splice variant sPD-L1, and other extracellular vesicles. sPD-L1 and evPD-L1 can bind PD-L1 inhibitors and outcompete PD-1 inhibitors, downregulating anti-tumor immunity and reducing or preventing response to PD-(L)1 inhibition (see, e.g., Chen et al., Nature, 560(7718):382-386, 2018; Mahoney et al., Cancer Immunol, Immunother, 1-12, 2018; Orme et al., supra; Poggio et al., Cell, 177(2):414-427.e13, 2019; Ricklefs et al., Science Adv, 4(3):eaar2766, 2018; Romero et al., Cancer Immunol, Immunother, 69(1):43-55, 2020; Theodoraki et al., Oncoimmunol, 8(7):e1593805, 2018; and Zhou et al., supra).

A representative full length amino acid sequence for human PD-L1 is set forth in SEQ ID NO:1:

MRIFAVFIFMTYWHLLNAFTVTVPKDLYVVEYGSNMTIECKFPVE
KQLDLAALIVYWEMEDKNIIQFVHGEEDLKVQHSSYRQRARLLKDQLSLG
NAALQITDVKLQDAGVYRCMISYGGADYKRITVKVNAPYNKINQRILVVD
PVTSEHELTCQAEGYPKAEVIWTSSDHQVLSGKTTTTNSKREEKLFNVTS
TLRINTTTNEIFYCTFRRLDPEENHTAELVIPELPLAHPPNERTHLVILG
AILLCLGVALTFIFRLRKGRMMDVKKCGIQDTNSKKQSDTHLEET (SEQ
ID NO:1)

The methods provided herein can be used to reduce the level of extracellular PD-L1, thus boosting the effectiveness of immunotherapies. In general, the methods provided herein include the use of TPE to reduce circulating extracellular PD-L1 in mammals identified as having cancer. In some cases, the mammal(s) can be identified as having reduced anti-tumor immunity based on the presence of a substrate (e.g., a PD-L1-related marker such as sPD-L1 or evPD-L1) at a level that is above or below (e.g., equal to or above) a predetermined threshold. The methods provided herein also can include administering immunotherapy to one or more mammals identified as having cancer and as having reduced anti-tumor immunity based on the presence of a substrate (e.g., a PD-L1-related marker such as sPD-L1 or evPD-L1) at a level that is above or below (e.g., equal to or above) a predetermined threshold, where the mammal(s) is/are subjected to TPE before administration of the immunotherapy.

The methods described herein can be used for treatment of any appropriate mammal (e.g., a human, non-human primate, horse, cow, pig, sheep, goat, cat, rabbit, guinea pig, hamster, rat, gerbil, or mouse) identified as being in need thereof. For example, a mammal can be identified as having a cancer (e.g., a tumor, malignancy, or otherwise abnormally proliferating cells). In some cases, the methods described herein can be used to treat mammals having a cancer in which the level of a PD-L1-related marker (e.g., sPD-L1, evPD-L1, or ADAM10/ADAM17-cleaved sPD-L1 ectodomain) is elevated. Cancers that may have elevated levels of one or more PD-L1 markers and can therefore be treated using the methods described herein include, without limitation, melanoma (e.g., metastatic melanoma), renal cancer, lung cancer (e.g., non-small cell lung cancer; NSCLC), mesothelioma, squamous cell cancer, a hematological cancer (e.g., leukemia or lymphoma, such as Hodgkin’s lymphoma), neurological cancer, breast cancer, prostate cancer, head and neck cancer, gastrointestinal cancer, liver cancer, pancreatic cancer, genitourinary cancer, bone cancer, bladder cancer, and vascular cancer.

A cancer that is resistant to immunotherapy in a subject (e.g., a mammal) may not respond to checkpoint inhibitors (e.g., inhibitors of PD-1/PD-L1 interactions) in an effective manner. In the methods provided herein, the subject in need of treatment can be a mammal identified as having an anti-PD-1 resistant or anti-PD-L1 resistant malignancy; in some cases, the methods provided herein can include identifying a subject as having an anti-PD-1 resistant or anti-PD-L1 resistant cancer. In some cases, the methods provided herein can include identifying a subject as having an immune system that is in some way unable to respond to anti-PD-1 or anti-PD-L1 treatment. A subject in need of the methods provided herein can be identified based on, for example, detection of sPD-L1, evPD-L1, or ADAM10/ADAM17-cleaved sPD-L1 ectodomain in a biological fluid sample (e.g., blood, plasma, serum, or urine), measurement of an elevated level of sPD-L1 evPD-L1, or ADAM10/ADAM17-cleaved sPD-L1 ectodomain in a biological fluid sample, or a combination of methods that include assessing the presence or level of sPD-L1. Having the ability to identify mammals as having a tumor and/or immune system that is resistant to treatment with inhibitors of PD-1/PD-L1 interactions can allow those mammals to be properly identified and treated in an effective and reliable manner. For example, the treatments provided herein in which TPE is combined with immunotherapy can be used to treat patients identified as having a tumor resistant to inhibitors of PD-1/PD-L1 interactions. Thus, the methods provided herein can be used to determine which patients are more likely to benefit from checkpoint inhibitor treatment alone, and which patients are more likely to require additional treatment to reduce sPD-L1 levels or availability, in addition to treatment with a checkpoint inhibitor.

An elevated level of a substrate (e.g., a PD-L1 related marker, such as sPD-L1, evPD-L1, or ADAM10/ADAM17-cleaved sPD-L1 ectodomain) is any level that is greater than a corresponding reference level (also referred to herein as a threshold or “cutoff” level) for the substrate. An elevated level of sPD-L1, evPD-L1, or ADAM10/ADAM17-cleaved sPD-L1 ectodomain can be, for example, 3 to 5% greater, 5 to 10% greater, 10 to 20% greater, 20 to 50% greater, 50 to 100% greater, or more than 100% greater than the threshold level of sPD-L1, evPD-L1, or ADAM10/ADAM17-cleaved sPD-L1 ectodomain. In some cases, an elevated level of sPD-L1, evPD-L1, or ADAM10/ADAM17-cleaved sPD-L1 ectodomain can be a level that is at least 2 percent (e.g., at least 3, 4, 5, 6, 7, 8, 9, 10, 15, 20, 25, 30, 35, 40, 45, 50, 55, 60, 65, 70, 75, 80, 85, 90, 95, 100, 200, 300, 400, or 500 percent) greater than the threshold level.

A threshold level for a substrate (e.g., a PD-L1-related marker such as sPD-L1, evPD-L1, or ADAM10/ADAM17-cleaved sPD-L1 ectodomain) in a mammal can be the level of the substrate that indicates a prognosis for the mammal (e.g., OS or time to recurrence). As described herein, a threshold value can be determined by measuring the level of a particular marker in samples from a population of mammals having different disease outcomes, and determining a level of the marker that can serve as a cutoff -where levels above the cutoff indicate poor prognosis and levels below the cutoff indicate a better prognosis, for example (or vice versa, depending on the marker). For sPD-L1, for example, the threshold can be from about 0.001 ng/mL to about 20 ng/mL (e.g., about 0.001 to about 0.01 ng/mL, about 0.01 to about 0.05 ng/mL, about 0.05 to about 0.1 ng/mL, about 0.1 to about 0.2 ng/mL, about 0.2 ng/mL to about 0.3 ng/mL, about 0.3 ng/mL to about 0.5 ng/mL, about 0.5 ng/mL to about 1 ng/mL, about 1 ng/mL to about 1.5 ng/mL, or about 1.5 ng/mL to about 2 ng/mL, about 2 ng/mL to about 5 ng/mL, about 5 ng/mL to about 10 ng/mL, about 8 ng/mL to about 12 ng/mL, or about 10 to about 20 ng/mL). In some cases, the threshold value for sPD-L1 can be from about 0.27 ng/mL to about 1.75 ng/mL, or about 0.277 ng/mL. In some cases, the threshold value for sPD-L1 can be from about 5 ng/mL to about 10 ng/mL, or about 7.5 ng/mL. In some cases, the threshold for evPD-L1 or evADAM10 can be from about 0.1% to 1% of circulating EVs (e.g., from about 0.1×10⁶ to about 0.4×10⁶ particles/uL, about 0.4×10⁶ to about 0.8×10⁶ particles/uL, about 0.5×10⁶ to about 1×10⁶ particles/uL, about 1×10⁶ to about 2×10⁶ particles/uL, or about 2×10⁶ to about 1.2x10⁷ particles/uL).

Any appropriate method can be used to detect and quantify substrates that are proteins or other macromolecules (e.g., PD-L1, sPD-L1, or ADAM10/ADAM17 cleaved sPD-L1 ectodomain). Suitable methods include, without limitation, enzyme-linked immunosorbent assay (ELISA), immunohistochemistry (IHC), and other antibody-based detection methods. In addition, any appropriate method can be used to detect substrates that include EVs (e.g., evPD-L1). “EVs” include extracellular vesicles, exosomes, and other structures for inter-cellular transport. Suitable methods for detecting and/or quantifying EVs include, without limitation, flow cytometry (e.g., nanoflow cytometry) and microscopic imaging techniques [e.g., electron magnetic imaging (EM)].

In some cases, the methods provided herein can include detecting and/or quantifying sPD-L1 in a sample of body fluid using, for example, immunological techniques. For example, an antibody that binds to an epitope specific for sPD-L1 can be used to detect sPD-L1 in body fluid. In some cases, an antibody directed against sPD-L1 can bind the polypeptide with an affinity of at least 10⁻⁴ M (e.g., at least 10⁻⁵, 10⁻⁶, 10⁻⁷, 10⁻⁸, 10⁻⁹, 10⁻¹⁰, 10⁻¹¹, or 10⁻¹² M).

Antibodies having specific binding affinity for sPD-L1 can be commercially obtained, or can be produced using, for example, methods described elsewhere (see, for example, Dong et al., Nature Med, 8:793-800, 2002). In some cases, a sPD-L1 polypeptide (e.g., a polypeptide comprising or consisting of the extracellular domain of PD-L1) can be recombinantly produced, or can be purified from a biological sample, and used to immunize a host animal such as, without limitation, a rabbit, chicken, mouse, guinea pig, or rat. Various adjuvants that can be used to increase the immunological response depend on the host species and include Freund’s adjuvant (complete and incomplete), mineral gels such as aluminum hydroxide, surface-active substances such as lysolecithin, pluronic polyols, polyanions, peptides, oil emulsions, keyhole limpet hemocyanin and dinitrophenol. Monoclonal antibodies can be prepared using a sPD-L1 polypeptide and hybridoma technology.

In immunological assays, an antibody having specific binding affinity for sPD-L1 or a secondary antibody that binds to such an antibody can be labeled, either directly or indirectly. Suitable labels include, without limitation, radioisotopes (e.g., ¹²⁵I, ¹³¹I, ³⁵S, ³H, ³²P, ³³P, or ¹⁴C), fluorophores (e.g., fluorescein, fluorescein-5-isothiocyanate (FITC), PerCP, rhodamine, or phycoerythrin), luminescent moieties (e.g., QDOT™ nanoparticles supplied by the Quantum Dot Corporation, Palo Alto, CA), compounds that absorb light of a defined wavelength, or enzymes (e.g., alkaline phosphatase or horseradish peroxidase). In some cases, antibodies can be indirectly labeled by conjugation with biotin and then detected with avidin or streptavidin labeled with a molecule described above. Methods of detecting or quantifying a label depend on the nature of the label, and can include, for example, the use of detectors such as x-ray film, radioactivity counters, scintillation counters, spectrophotometers, colorimeters, fluorometers, luminometers, and densitometers. Combinations of these approaches (including “multi-layer” assays) can be used to enhance the sensitivity of an assay.

Immunological assays for detecting sPD-L1 can be performed in a variety of formats, including sandwich assays (e.g., ELISA assays, sandwich Western blotting assays, or sandwich immunomagnetic detection assays), competition assays (competitive RIA), or bridge immunoassays. See, for example, U.S. Pat. Nos. 5,296,347; 4,233,402; 4,098,876; and 4,034,074. Methods of detecting sPD-L1 generally can include contacting a body fluid with an antibody that binds to sPD-L1 and detecting or quantifying binding of sPD-L1 to the antibody. For example, an antibody having specific binding affinity for sPD-L1 can be immobilized on a solid substrate and then exposed to the biological sample. In some cases, binding of sPD-L1 to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB; Rapsgatan, Sweden). Alternatively, the antibody can be labeled and detected as described above. A standard curve using known quantities of sPD-L1 can be generated to aid in the quantitation of sPD-L1 levels.

In some embodiments, a “sandwich” assay in which a capture antibody or capture binding substrate is immobilized on a solid substrate can be used to detect the presence, absence, or amount of sPD-L1. The solid substrate can be contacted with the biological sample such that sPD-L1 in the sample can bind to the immobilized antibody. The presence of sPD-L1 bound to the antibody can be determined using a “reporter” antibody having specific binding affinity for sPD-L1 and the methods described above. It is understood that in these sandwich assays, the capture antibody or capture binding substrate (e.g., an immobilized PD-1 receptor fragment) should not bind to the same epitope (or range of epitopes in the case of a polyclonal antibody) as the reporter antibody. Thus, if a monoclonal antibody is used as a capture antibody, the reporter antibody can be another monoclonal antibody that binds to an epitope that is either completely physically separated from or only partially overlaps with the epitope to which the capture monoclonal antibody binds, or a polyclonal antibody that binds to epitopes other than or in addition to that to which the capture monoclonal antibody binds.

Suitable solid substrates to which an antibody (e.g., a capture antibody) or capture binding substrate can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be used when an automated immunoassay system is used.

Alternative techniques for detecting sPD-L1 include mass-spectrophotometric techniques such as electrospray ionization (ESI), liquid chromatography-mass spectrometry (LC-MS), and matrix-assisted laser desorption-ionization (MALDI). See, for example, Gevaert et al., Electrophoresis, 22(9):1645-51, 2001; and Chaurand et al., J Am Soc Mass Spectrom, 10(2):91-103, 1999). Mass spectrometers useful for such applications are available from Applied Biosystems (Foster City, CA); Bruker Daltronics (Billerica, MA) and Amersham Pharmacia (Sunnyvale, CA). Arrays for detecting polypeptides, two-dimensional gel analysis, and chromatographic separation techniques also can be used to detect sPD-L1.

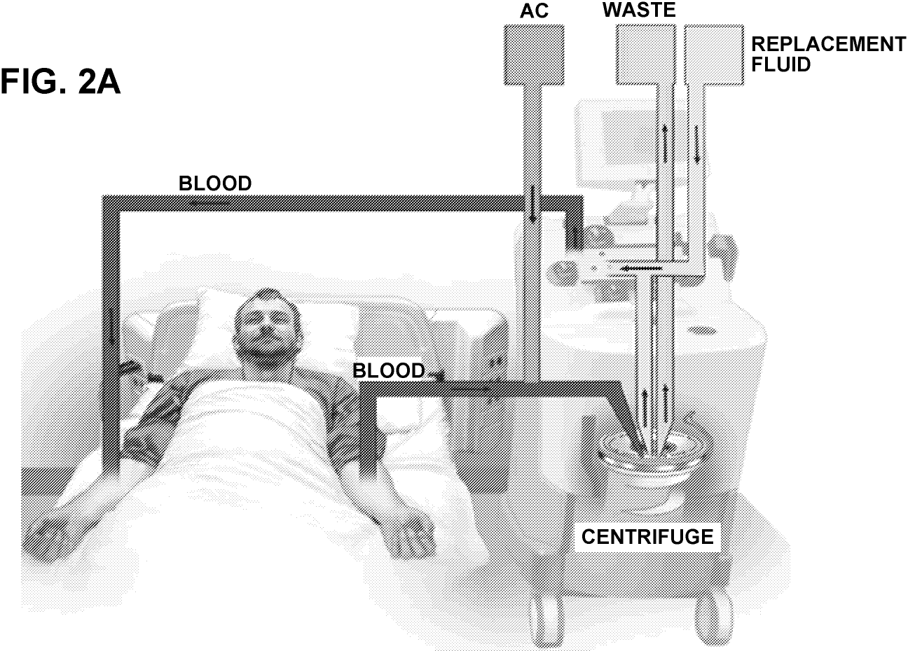

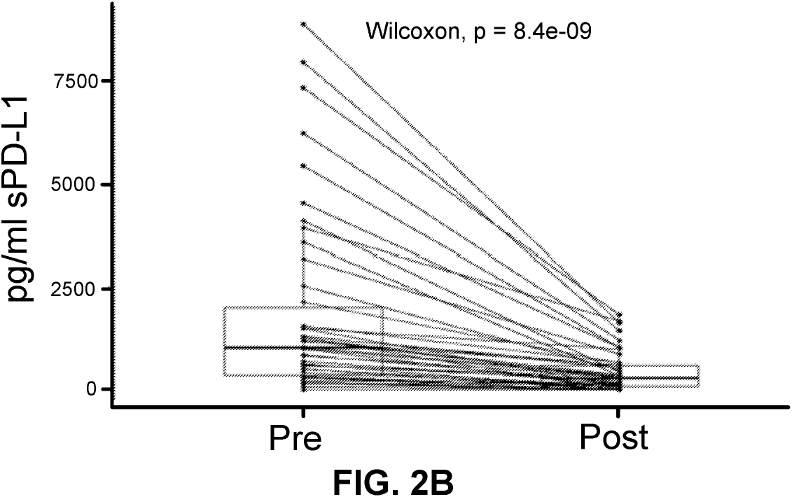

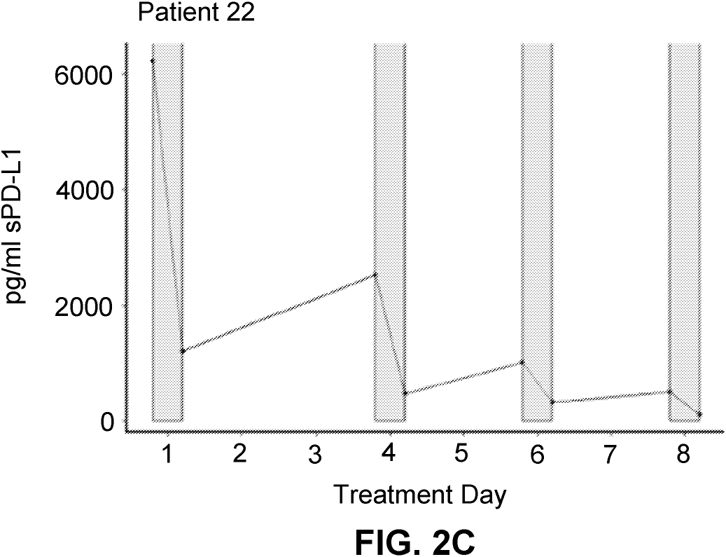

When a subject is identified as having a level of a substrate (e.g., a PD-L1 related marker such as sPD-L1 or evPD-L1) that is at or above a threshold level, the subject can undergo one or more (e.g., one, two, three, four, five, one to three, two to four three to five, or more than five) TPE procedures. TPE (depicted in FIG. 2A) also is referred to as plasmapheresis or apheresis, and is a procedure in which blood is removed from a subject, the plasma is removed and replaced with another fluid, and the remaining blood components and replacement fluid are returned to the subject. The result is that noncellular substances confined to the plasma are removed from the subject. In some cases, the methods provided herein can include determining the level of the substrate (e.g., the PD-L1 related marker) in the blood before and/or after each round of TPE, thus permitting the level of the substrate to be monitored until it crosses the threshold level. In some cases, TPE can be repeated until the level of the substrate is reduced by at least 10% (e.g., at least 15%, at least 20%, at least 50%, 10% to 15%, 15% to 20%, 20% to 25%, 25% to 50%, or more than 50%) as compared to a previously measured level of the substrate (e.g., sPD-L1) in a sample obtained from the subject before or during treatment (e.g., at an earlier time point during a TPE procedure).

After the TPE step(s) is complete, the subject can be treated with immunotherapy. The immunotherapy can be include one or more immune checkpoint inhibitors targeted to an immunomodulatory receptor (e.g., PD-1 or CTLA-4) or an immunomodulatory ligand (e.g., PD-L1, PD-L2, PD-L3, CD80, or CD86) that binds to an immunomodulatory receptor. Checkpoint inhibitors (e.g., inhibitors of PD-1/PD-L1 interaction) that can be used in the methods provided herein include, without limitation, anti-PD-1 antibodies, anti-PD-L1 antibodies, anti-CTLA-4 antibodies, and any combination thereof (e.g., nivolumab and ipilimumab). Anti-PD-1, anti-PD-L1, and anti-CTLA-4 antibodies that can be used as described herein can be polyclonal antibodies, monoclonal antibodies, humanized antibodies, chimeric antibodies, single chain Fv antibody fragments, Fab fragments, or F(ab)2 fragments that are capable of binding to an epitopic determinant of PD-1 (e.g., human PD-1), PD-L1 (e.g., human PD-L1), or CTLA-4 (e.g., human CTLA-4). Examples of anti-PD1 antibodies that can be used as described herein include, without limitation, pembrolizumab (a humanized antibody with the trade name KEYTRUDA®, available from Merck), nivolumab (a targeted antibody with the trade name OPDIVO®, available from Bristol-Myers Squibb), and pidilizumab (a monoclonal antibody available from Medivation). Examples of anti-PD-L1 antibodies that can be used as described herein include, without limitation, avelumab (a monoclonal antibody with the trade name BAVENCIO®, available from Pfizer), atezolizumab (a humanized monoclonal antibody with the trade name TECENTRIQ®, available from Genentech) and durvalumab (a monoclonal antibody with the trade IMFINZI®, available from AstraZeneca). Examples of anti-CTLA-4 antibodies that can be used in the methods described herein include, without limitation, ipilimumab (a monoclonal antibody with the trade name YERVOY®, available from Bristol-Myers Squibb).

A immunotherapeutic composition containing, for example, one or more inhibitors of PD-⅟PD-L1 interaction (e.g., one or more anti-PD-1 antibodies, one or more anti-PD-L1 antibodies, or a combination thereof) can be administered to a subject in an amount, at a frequency, and for a duration effective to achieve a desired effect (e.g., to reduce tumor size, reduce cancer cell number, to reduce one or more symptoms of cancer, or to prevent or delay worsening of one or more such symptoms). The immunotherapy (e.g., one or more inhibitors of PD-⅟PD-L1 interaction) can be administered in an amount effective to reduce the size of a tumor, reduce the number of cancer cells, or reduce one or more symptoms of cancer in a patient by at least 3% (e.g., at least 5%, at least 10%, at least 20%, at least 50%, 3% to 5%, 5% to 10%, 10% to 15%, 15% to 20%, 20% to 25%, 25% to 50%, or more than 50%). In some cases, for example, effective amount of an immunotherapy treatment can be an amount that reduces the size of a tumor in a treated mammal by at least 10% as compared to the size of the tumor in the mammal prior to administration of the immunotherapy. The presence or extent of tumors, cancer cells, and cancer symptoms can be evaluated using any appropriate method.

In some embodiments, the amounts of one or more immunotherapies (e.g., one or more inhibitors of PD-1/PD-L1 interactions) administered to a mammal and/or the frequency of administration can be titrated in order to, for example, identify a dosage that is most effective to treat the mammal while having the least amount of adverse effects. For example, an effective amount of a composition containing one or more inhibitors of PD-1/PD-L1 interaction can be any amount that reduces tumor size or reduces cancer symptoms within a mammal, without having significant toxicity in the mammal. If a cancer in a mammal fails to respond to a particular amount, then the amount can be increased by, for example, two-fold, three-fold, five-fold, or ten-fold. After receiving this higher concentration, the mammal can be monitored for both responsiveness to the treatment and toxicity symptoms, and adjustments in the dosage can be made accordingly. The effective amount can remain constant or can be adjusted as a sliding scale or variable dose depending on the mammal’s response to treatment.

In some embodiments, the methods provided herein can include monitoring a treated subject to determine whether or not the therapy is effective. For example, a mammal having a tumor (e.g., a human cancer patient) can be monitored to determine whether the tumor has decreased in size after treatment, or whether the number of tumor cells detected in the patient is reduced following treatment.

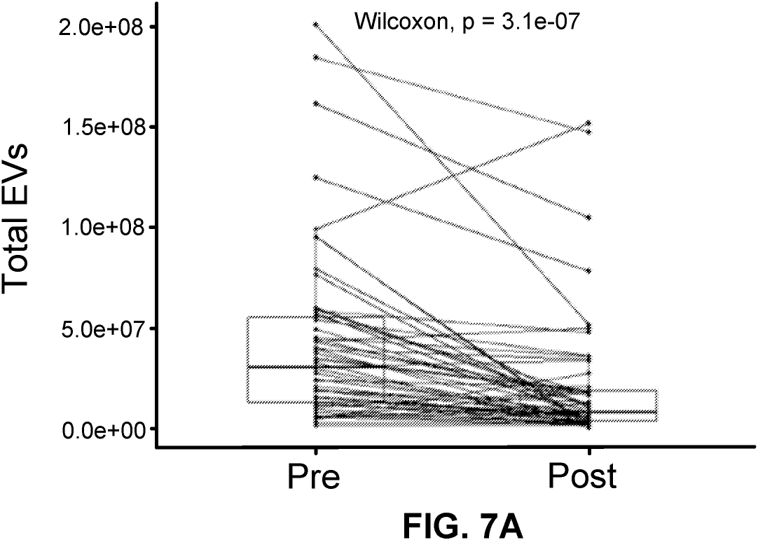

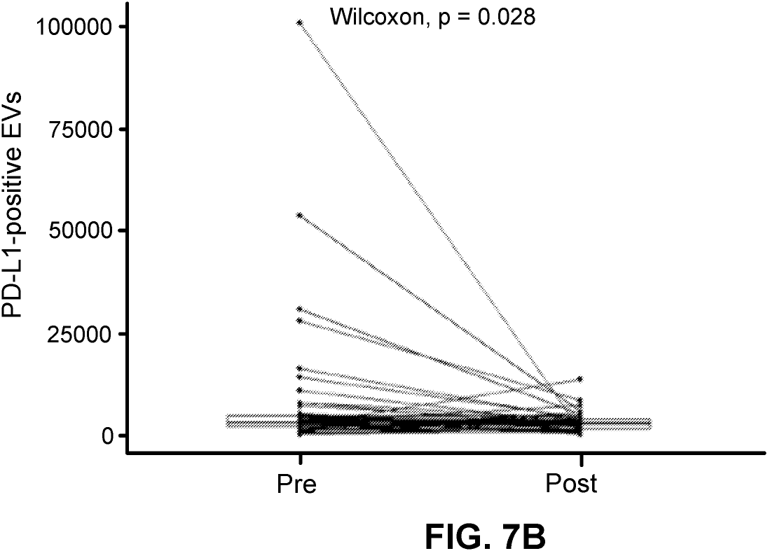

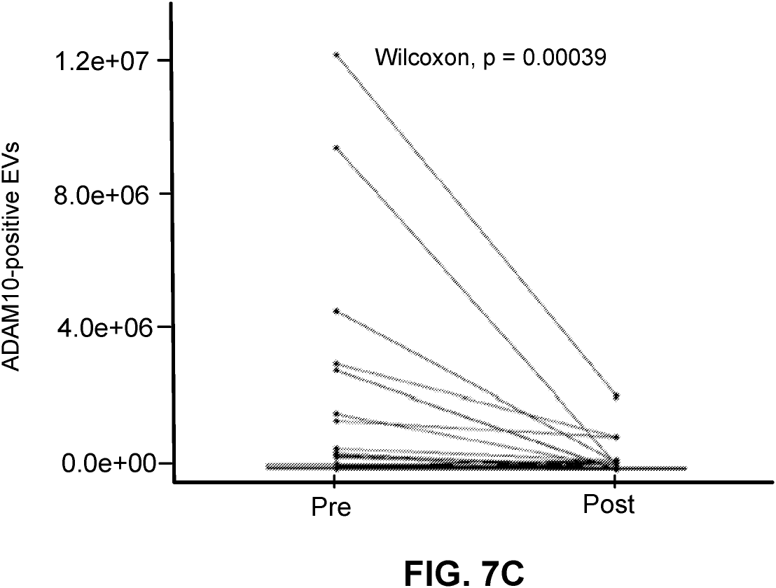

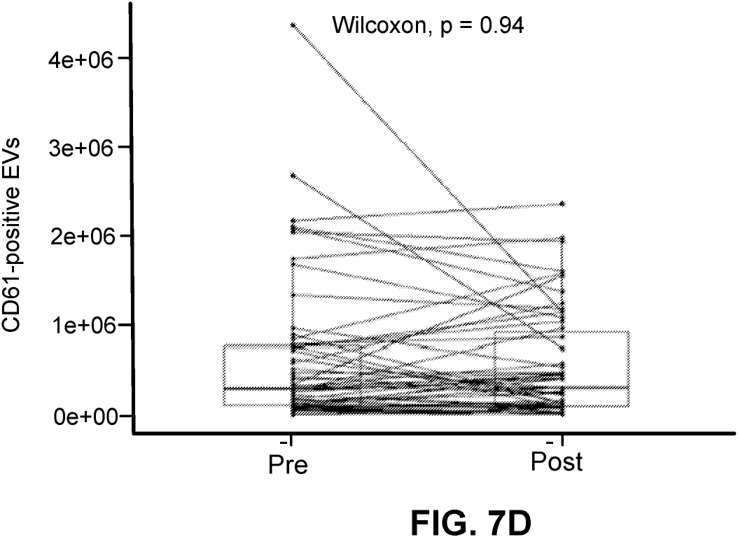

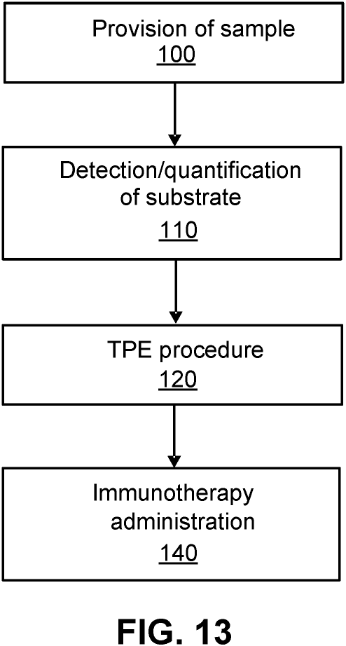

In some cases, a method as described herein can include the steps shown in FIG. 13. The depicted method includes providing a biological sample from a mammal (100), detecting or quantifying a target substrate (110), conducting TPE to reduce the level of the target substrate in the mammal (120), and administering an immunotherapeutic agent (130) to the mammal. In some cases, the methods provided herein can include only steps 120 and 130. For example, a method can include conducting TPE on a mammal identified as having a level of a target substrate (also referred to herein as a PD-L1-related marker) that is equal to or greater than a threshold level, and then administering an immunotherapy to the mammal. The methods described herein can be surprisingly effective, as no clinical intervention has previously been shown to eliminate sPD-L1, evPD-L1, or EVs from a subject. The materials and methods provided herein therefore are useful because they can improve the ability of PD-(L)1 inhibitors to reach their intended receptors. In addition, it is noteworthy that TPE does not remove all substances confined to the plasma, as evidenced by unchanging evCD61 levels before and after TPE as described below and shown in FIG. 7D, for example (see, also, Padmanabhan et al., J Clin Apheresis, 34(3):171-354, 2019). Thus, it would not be expected that these particular immunosuppressive, immunotherapy-thwarting substances would be removed by any such mechanical process.

It is to be noted that this document also provides methods for removing EVs to address other clinical indications including, but not limited to, aging, autoimmunity, heart disorders, infection, neurodegeneration, and obesity.

The invention will be further described in the following examples, which do not limit the scope of the invention described in the claims.

### EXAMPLES

### Example 1 - Materials and Methods

Retrospective Melanoma Outcomes Study Design: In a retrospective analysis, baseline blood samples from 276 patients with melanoma prior to treatment in one of three clinical trials by the North Central Cancer Treatment Group (NCCTG): N057e (Kottschade et al., Cancer, 117(8):1704-1710, 2011), N0775 (ottschade et al., KCancer, 119(3):586-592, 2013), and N0879 (McWilliams et al., Cancer, 124(3):537-545, 2018) between 2006 and 2014 were tested for sPD-L1. 82% of patients were diagnosed with cutaneous melanoma, and none received immunotherapy treatments. Blood from 86 healthy volunteers undergoing blood donation at Mayo Clinic also was tested.

Prospective Therapeutic Plasma Exchange Study Design: Adult subjects undergoing TPE were identified for an investigator-initiated open-label single-center observational study. Samples of whole blood were collected in ACD vacutainers (BD) immediately prior to TPE and upon completion of the procedure. In each case, the first 8 mL of blood was discarded to avoid contamination, after which an 8 mL sample was obtained in sequence. Plasma was isolated by centrifugation. A post-procedure blood sample was obtained after completion of the procedure. In addition, matching samples from discarded plasma from the procedure waste bag were collected. Samples were obtained from up to four consecutive procedures for each patient. If a patient underwent fewer than four TPE procedures, samples were obtained from as many procedures as possible.

The patients included in the study were adults undergoing TPE for a variety of hematologic, neurologic, and renal diseases as indicated by published guidelines from the American Society for Apheresis (ASFA) or according to the medical judgement of the referring physicians (Padmanabhan et al., J Clin Apher, 34(3):171-354, 2019). Patients taking biotin supplements were excluded from the study due to biotin interference with the sPD-L1 ELISA assay. Procedures were performed using centrifugation-based cell separators, either the Fenwal Amicus (Fresenius KABI USA LLC, Lake Zurich IL, USA) or the Spectra Optia (Terumo BCT Inc, Lakewood CO, USA). For each patient, a single plasma volume was exchanged using either peripheral IVs (preferred) or central lines for vascular access. For this study, due to the possibility of sPD-L1 or PD-L1-positive EVs present in donor plasma, only TPE sessions using no donor plasma (i.e., no FFP) in the replacement fluid were included in calculations. Anticoagulation consisted of either 500 ml of acid citrate dextrose solution A (ACD-A) or 500 ml of ACD-A with 5000 units of unfractionated heparin. Anticoagulant to blood ratios were 1:13 when ACD-A was used and 1:26 when ACD-A/Heparin was used. Patients did not receive routine electrolyte replacement, but 10 ml of 10% calcium gluconate was administered by slow IV push for signs and symptoms of hypocalcemia related to the ACD-A anticoagulant in one patient.

ELISA: Enzyme-linked immunosorbent assay (ELISA) was performed as described elsewhere (Frigola et al., Clin Cancer Res 1(177):1915-1923, 2011). Both secreted splice variant and shed sPD-L1 are reliably detected by this ELISA. In brief, paired mouse IgG2 monoclonal antibody clones H1A and B11 against extracellular human PD-L1 were utilized in a capture-detection plate assay using biotinylation and HRP-streptavidin detection. This assay is specific for sPD-L1 and does not exhibit cross-reactivity to other B7-H homologues, nor to evPD-L1. Concentrations were determined by optical density (OD) measurements along a standard curve of recombinant human PD-L1. ELISAs were performed by team members blinded to the identity of the samples.

Flow cytometry: Flow cytometry for EVs was performed as described elsewhere (Gomes et al., Thromb Haemost 118(9):1612-1624, 2018). In brief, plasma samples were centrifuged twice at 2000 g to deplete platelets. Resultant platelet-free plasma (PFP) was analyzed using an A60-Micro Plus Nanoscale Flow Cytometer (Apogee FlowSystems Ltd., Hemel Hempstead, England) gating for mid-intensity light angle light scatter (LALS) and markers of interest. Anti-PD-L1 (atezolizumab; Genentech, South San Francisco, CA), ADAM10 (clone 163003; R&D Systems/Bio-Techne, Minneapolis, MN), and CD61 (clone VI-PL2; BioLegend, San Diego, CA) antibodies were conjugated to fluorophores (Alexa-647, PE phycoerythrin, and Alexa-488; Life Technologies/Thermo Fisher Scientific, Waltham, MA) and titrated prior to use. Nanoscale flow cytometer calibration was performed using a standard reference bead mix as described elsewhere (Gomes et al., supra). Flow cytometry was performed by team members blinded to the identity of the samples.

Statistical Analysis: All statistical analyses were performed using R Statistical Software (R Foundation). Retrospective progression-free survival (PFS) was analyzed using Kaplan-Meier and Cox proportional-hazards modeling. Optimal cutoff values for sPD-L1 levels were determined using the greyzoneSurv package for R. Wilcoxon signed-rank test was used to compare paired pre- and post-TPE patient sample sPD-L1 and EV levels as indicated. Baseline clinical characteristics for the study were compared by Kruskal-Wallis test for continuous variables and Pearson’s chi-squared test for discrete variables as indicated. Otherwise, groups were compared by unpaired two-sided Student’s t-test. Figures containing box plots show quartile values and individual data points. Mean values and 95% confidence intervals (CI) are indicated in corresponding supplemental figures and tables. P <0.05 was considered statistically significant. In the figures presented herein, p values are denoted <0.05 with *, <0.01 with **, and <0.001 with ***.

### Example 2 - Soluble PD-L1 Levels Predict Overall Survival in Patients With Melanoma

Each form of extracellular PD-L1 acts in trans as a systemic immunosuppressant through PD-1 signaling (FIG. 1A, TABLE 1) (Zhou et al., supra; Mahoney et al., Cancer Immunol Immunother, 68:421-432, 2019; Orme et al., supra; Romero et al., Cancer Immunol Immunother, 69(1):43-55, 2020; Chen et al., Nature, 560(7718):382-386, 2018; and Poggio et al., Cell, 177(2):414-427, 2019). To confirm the clinical impact of plasma sPD-L1, sPD-L1 levels were measured in a retrospective cohort of 276 patients with melanoma. Exploratory analysis of overall survival (OS) determined a working cutoff value of sPD-L1 (≥ 0.277 ng/mL) and baseline characteristics at the time of entry into study were similar (TABLE 2). Patients with high plasma sPD-L1 levels experienced inferior median OS compared with patients with low plasma sPD-L1 levels (FIG. 1B, 12.1 vs 18.5 months, p=0.005). In comparison to age-matched controls, patients with melanoma exhibited higher mean plasma sPD-L1 (FIG. 1C, 1.72 ng/mL vs. 0.77 ng/mL, p<0.001). In a multivariate Cox proportional hazards analysis, high sPD-L1 prior to treatment predicted worse survival (HR 1.49; 95% CI 1.06-2.09; p=0.025) when accounting for advanced age (not significant), sex (not significant), late stage (p=0.002) and high serum LDH (p=0.01) (TABLE 3).

TABLE 1

Extracellular forms of PD-L1 and outcomes in multiple malignancies

| | | | | |
|---|---|---|---|---|
| | High sPD-L1 prognosis | PD-L1 protein-to-mRNA prognosis¹ | First Line IO | Non-First Line IO |
| Adrenocortical | | ↑ | | |
| Bladder | | ↑ | Cisplatin-ineligible CPS10: P²A³ | P² N⁴ |
| Breast | Poor⁵ | ↑ | TNBC unresectable PD-L1>1%: A³ | |
| Biliary | Poor ⁶ | | | |
| Cervical | | ↗ | | P² |
| Colon | | | MSI-high: P² | |
| Gastric | Mainly poor⁷⁻¹¹ Poor in IO¹² Poor with EVs¹³ | ↓ | | P² |
| Glioma (low-grade) | | ↑ | | |
| Head/Neck SCC | | ↗ | P² | N⁴ |
| Hepatocellular | Poor DFS and OS¹⁴⁻¹⁸ or equivocal¹⁹ | ↑ | | P² N⁴ |
| Lung | Poor²⁰⁻²³ Poor response in IO^{12,24,25} | | Metastatic NSCLC no muts: P²A³ SCLC: A³ | P² N⁴ |
| Lymphoma | BCL poor OS²⁶ poor response²⁷ decrease on tx positive²⁸ HL poor PFS²⁹ PTCL poor OS/PFS/response^{30,31} NK/T poor OS/PFS^{32,33} | ↗ | | HL: P² N⁴ |
| Melanoma | Poor34 Poor in IO³⁵ | ↗ | Unresectable/metastatic: P²N⁴ | |
| | | | Adjuvant if LNs after resection: P² | |
| Mesothelioma | | ↑ | | |
| Myeloma | Poor PFS^{36,37} | | | |
| Ovarian (epithelial) | Poor OS and PFS in cisplatin-resp³⁸ | | | |
| Pancreatic | Decrease on tx positive³⁹ | | | |
| Prostate | | ↗ | | |
| Rectal | Poor DFS⁴⁰ | | | |
| Renal | | ↗ | Advanced with axitinib: P² | N⁴ |
| Sarcoma | | ↑ | | |

Cancers for which serum sPD-L1 and/or PD-L1 protein-to-mRNA ratios affect(s) prognosis in a previous study of Cancer Genome Atlas (TCGA) data (increased improves survival)1 and/or in which first-line or common second-line PD-(L)1 inhibitor therapy is FDA approved as a partial list. OS: overall survival. PFS: progression-free survival. IO: immunotherapy. A: atezolizumab. N: nivolumab. P: pembrolizumab.

### REFERENCES

1. Orme et al., supra.
2. Keytruda: Highlights of Prescribing Information, 2017
3. Genentech. Atezolizumab: Highlights of Prescribing Information, 2018
4. Nivolumab: Highlights of Prescribing Information, 2018
5. Li et al., supra
6. Ha et al., supra
7. Zheng et al., Chin J Cancer Res, 26:104-111, 2014
8. Ito et al., Ann Gastroenterol Surg, 2(4):313-318, 2018
9. Bang et al., J Clin Oncol, 33:4001, 2015
10. Shigemori et al., Ann Surg Oncol, 26:876-883, 2019
11. Choi et al., Ann Surg, 270:309-316, 2019
12. Ando et al., supra
13. Fan et al., supra
14. Finkelmeier et al., Z Gastroenterol, 53:A4_19, 2015
15. El-Gebaly et al., Curr Cancer Drug Targets, 19(11):896-905, 2019
16. Chang et al., Cancer Immunol Immunother, 68:353-363, 2019
17. Han et al., J Cancer Res Clin Oncol, 145:303-312, 2019
18. Kim et al., Radiother Oncol, 129:130-135, 2018
19. Elmezayen et al., Surg Today, 50(6):569-576, 2019
20. Okuma et al. 2017, supra
21. Zhang et al., supra
22. Costantini et al., supra
23. Zhao et al., supra
24. Costantini et al., supra
25. Okuma et al. 2018, supra
26. Rossille et al., Leukemia, 28, 2367-2375, 2014
27. Rossille et al., supra
28. El-Ghammaz, et al., Clin Exp Med, 18:505-512, 2018
29. Guo et al., Transl Oncol, 11:779-785, 2018
30. Zhang et al., Hematol Oncol, 37:270-276, 2019
31. Shen et al., Hematol, 24:392-398, 2019
32. Bi et al., J Hematol Oncol, 9:109, 2016
33. Wang et al., Oncotarget 7:33035-33045, 2016
34. Dronca et al., supra
35. Zhou et al., supra
36. Wang et al., Oncotarget 6:41228-44136, 2015
37. Huang et al., Oncotarget 7:62490-62502, 2016
38. Buderath et al., Front Oncol, 9:1015, 2019
39. Park et al., Sci Rep, 9:11131, 2019
40. Tominaga et al., PLoS One, 14:e0212978, 2019

TABLE 2

Baseline patient characteristics in the melanoma cohort

| | High sPD-L1 (n=216) | Low sPD-L1 (n=61) | Statistic |
|---|---|---|---|
| Age, years | 60 (50, 69) | 58 (50, 70) | F=0.10, P = 0.756¹ |
| Sex: M | 137/216 (63) | 31/60 (52) | X² = 2.73, P=0.10² |
| Stage | | | X² = 3.95, P=0.139² |
| M1a | 31/214 (14.5) | 14/59 (23.7) | |
| M1b | 54/214 (25.2) | 17/59 (28.8) | |
| M1c | 129/214 (60.3) | 28/59 (47.5) | |
| sPD-L1, ng/uL | 0.589 (0.370, 3.153) | 0.216 (0.173, 0.249) | F=272.68 P < 0.001¹ |
| LDH, U/L | 209 (168, 395) | 187 (163, 298) | F=2.05 P = 0.154¹ |

Patients with melanoma from three studies with retrospective data were compared by starting sPD-L1 level above or below the discovered survival cutoff (0.277 ng/mL).
Characteristics collected at entry into the study included age in years, sex, stage, sPD-L1, and LDH (lactate dehydrogenase). ¹Kruskal-Wallis. ²Pearson

TABLE 3

Multivariate analysis in melanoma survival at diagnosis

| | Hazard Ratio | P value |
|---|---|---|
| Age > 60 years | 1.00 (0.76-1.32) | 0.93 |
| Sex: M | 1.19 (0.90-1.58) | 0.25 |
| Stage | | |
| M1a | 1 | |
| M1b | 1.42 (0.91-2.23) | 0.11 |
| M1c | 1.94 (1.28-2.96) | 0.002 ^{∗∗} |
| sPD-L1 > 0.277 ng/mL | 1.47 (1.05-2.07) | 0.025 ^{∗} |
| LDH > 180 U/L | 1.53 (1.53, 2.11) | 0.010 ^{∗∗} |

Multivariate analysis in melanoma survival at diagnosis. Cox proportional Hazards multivariate analysis was performed.

### Example 3 - Therapeutic Plasma Exchange Significantly Reduces Plasma sPD-L1 Levels

To determine whether TPE can remove extracellular PD-L1 in its various forms, patients undergoing planned TPE were prospectively enrolled. 28 patients met inclusion criteria, and 25 provided informed consent. Baseline patient characteristics are listed in TABLE 4. One patient was excluded for biotin-containing supplement use, as biotin interferes with the established sPD-L1 detection assay. The remaining 24 patients underwent plasma exchange and sample collection before and after the procedure as described in Example 1 and depicted in FIG. 2A. Discarded plasma samples from the TPE device waste bag for each session also were collected. sPD-L1 was measured in each sample, and most patients undergoing TPE exhibited sPD-L1 levels above the clinically relevant 0.277 ng/mL cutoff from the retrospective melanoma study.

TABLE 4

Patient baseline characteristics

| Characteristic | High sPDL1 (n=17) | Low sPDL1 (n=7) | Statistic |
|---|---|---|---|
| Starting sPD-L1 | 1650 (962, 6663) | 108 (0, 128) | F_{1.22} = 36.19, P < 0.001¹ |
| Age (years) | 61 (42, 69) | 47 (38, 66) | F_{1.22} = 0.35, P=0.558¹ |
| Gender: F | 8 (47%) | 2 (29%) | X² = 0.70, P=0.404² |
| Active Cancer: Yes | 5 (29%) | 2 (29%) | X² = 0, P=0.967² |
| Immunntherapy | | | X² = 2.88, P=0.237² |
| None | 16 (94%) | 6 (86%) | |
| Atezolizumab | 1 (6%) | 0 (0%) | |
| Pembrolizumab | 0 (0%) | 1 (14%) | |
| Plasma exchange indication | | | X² = 2.88, P=0.315² |
| CNS demyelination (myelitis, MS, NMO, myelopathy) | 5 (29%) | 5 (71%) | |
| Immune encephalitis | 2 (12%) | 0 (0%) | |
| Myasthenia gravis | 1 (5.9%) | 0 (0%) | |
| Paraneoplastic syndrome (encephalitis, neuropathy, pemphigus) | 2 (12%) | 2 (29%) | |
| Paraproteinemia (Waldenström, cryoglobulinemia, kappa gammopathy) | 3 (18%) | 0 (0%) | |
| Susac syndrome | 2 (12%) | 0 (0%) | |
| Transplant rejection (heart, kidney) | 2 (12%) | 0 (0%) | |
| Pre-TPE White Blood Cell Count | 6.7 (6.1, 10.3) | 8.8 (7.9, 12.3) | F_{1,22} = 0.78, P = 0.385¹ |
| Pre-TPE Hemoglobin | 12.5 (10.4, 13.5) | 14.7 (13.5, 14.9) | F_{1,22} = 8.60, P = 0.008¹ |
| Pre-TPE Creatinine | 0.90 (0.79, 1.55) | 0.710 (0.63, 0.89) | F_{1,22} = 3.82, P = 0.063¹ |

Patients undergoing TPE were compared by starting sPD-L1 level above or below a survival cutoff established in patients with melanoma (0.277 ng/mL). For categorical variables, n (%) is given. For continuous variables, mean (quartiles) is given. ¹Kruskal-Wallis. ²Pearson. MS, multiple sclerosis; NMO, neuromyelitis optica.

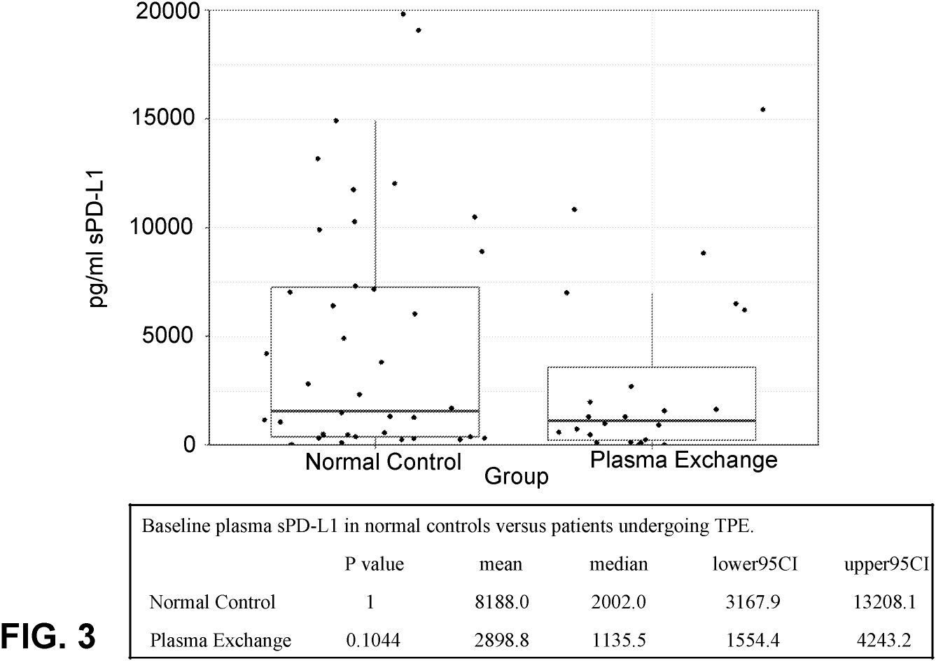

Most patients undergoing TPE did not have an active cancer diagnosis. Baseline sPD-L1 levels in all patients were compared to matched normal controls and patients with melanoma (FIG. 3), and some patients exhibited sPD-L1 above the clinically significant cutoff level determined in the retrospective melanoma cohort. Patients with high baseline sPD-L1 levels were significantly more anemic than patients with lower baseline sPD-L1 even when controlling for the higher number of female subjects in the high sPD-L1 group (female-only mean Hgb 11.4 vs 14.1, p=0.04; male-only mean Hgb 11.8 vs 14.3, p=0.03). Groups were otherwise similar. TPE significantly reduced plasma sPD-L1 levels in patients receiving albumin-only (i.e., no FFP) replacement fluid (FIG. 2B, p < 0.0001). Removed sPD-L1 was detected in matching plasma samples from the TPE procedure waste bag. Each TPE session removed a mean 70.8% of detectable plasma sPD-L1; mean regeneration of sPD-L1 between sessions was 33.8% (TABLE 5). TPE sessions usually were separated by one to three days.

TABLE 5

sPD-L1 reduction and regeneration per exchange

| % Reduction per exchange | (n=44) |
|---|---|
| Mean (SD) | 70.8 (21.3) |
| Median [Min, Max] | 74.4 [-5.10, 100] |
| % Regeneration between exchanges | (n=44) |
| Mean (SD) | 33.8 (84.1) |
| Median [Min, Max] | 45.5 [-429, 100] |
| Regeneration per cycle (pg/ml) | |
| Mean (SD) | 1250 (3300) |
| Median [Min, Max] | 466 [-3.8k, 15.4k] |

For each exchange not requiring FFP, percent sPD-L1 reduction and regeneration between each exchange was calculated. n=44.

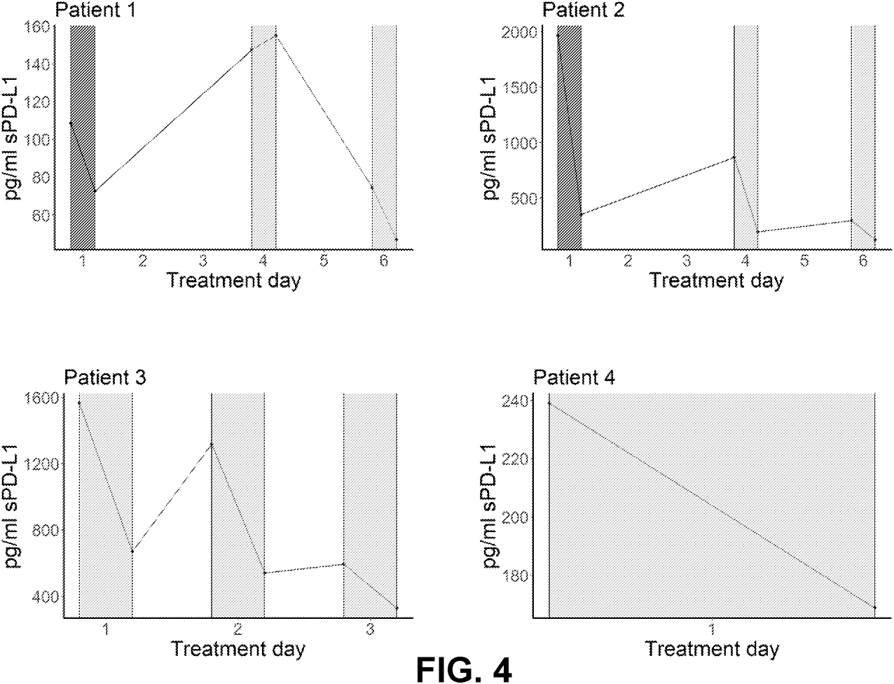

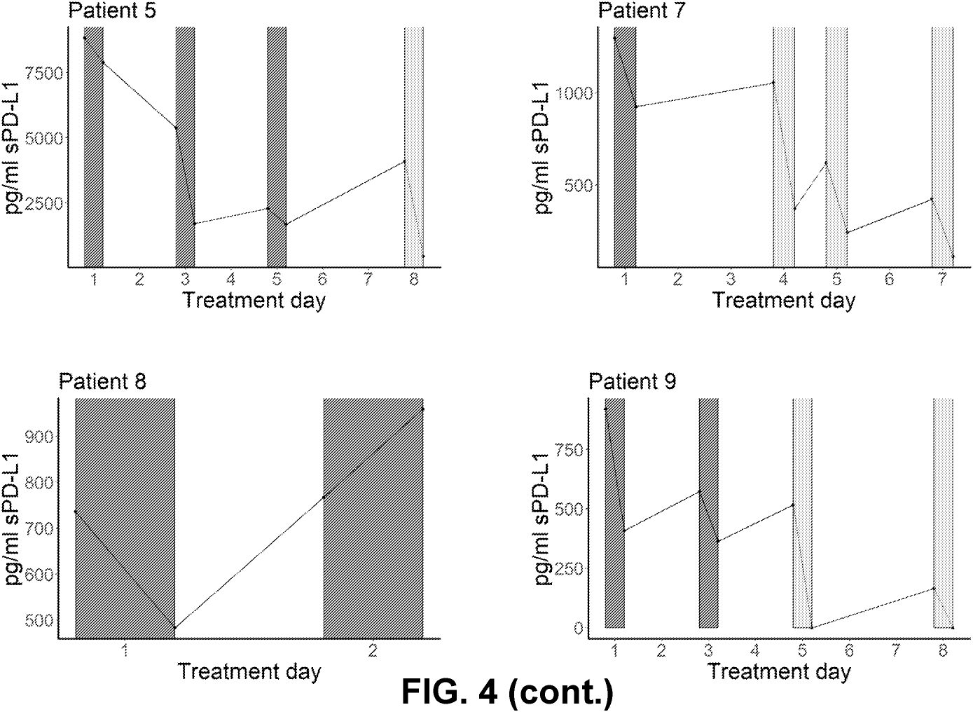

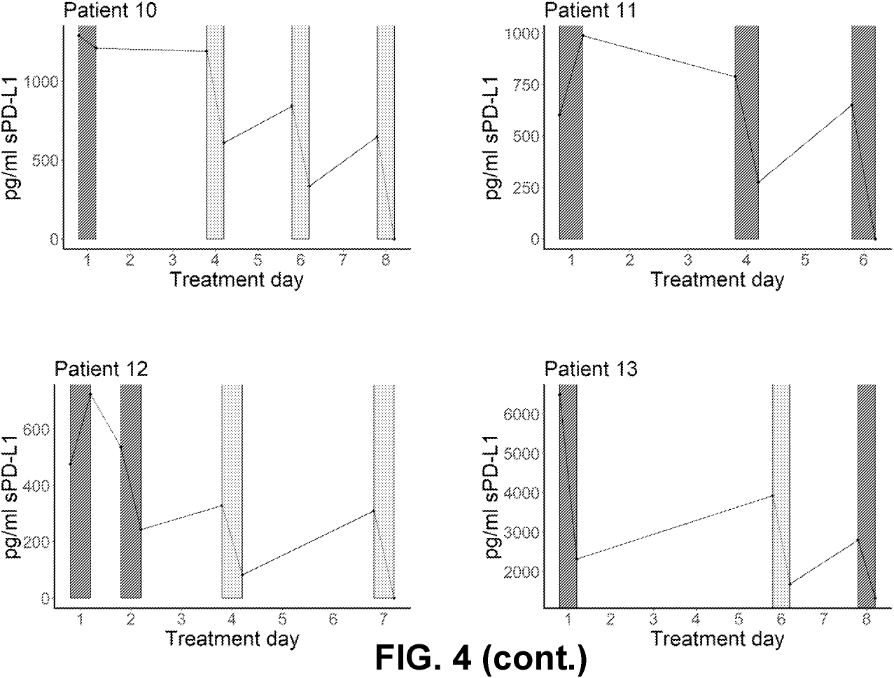

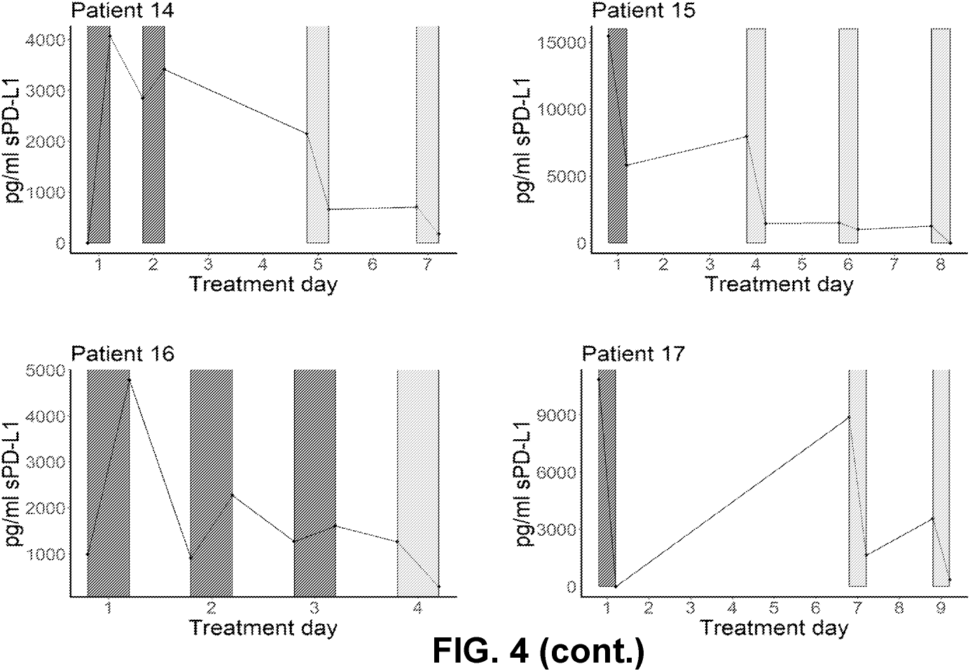

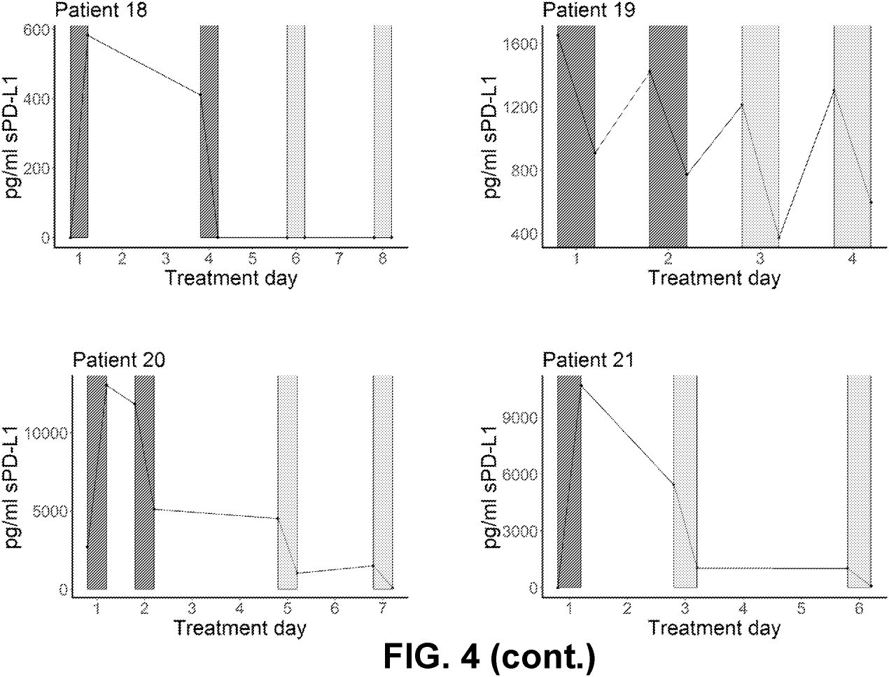

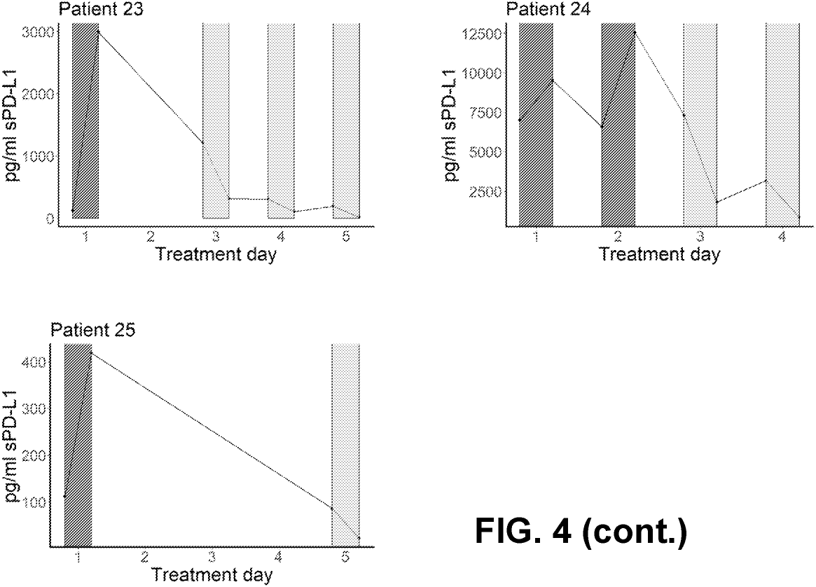

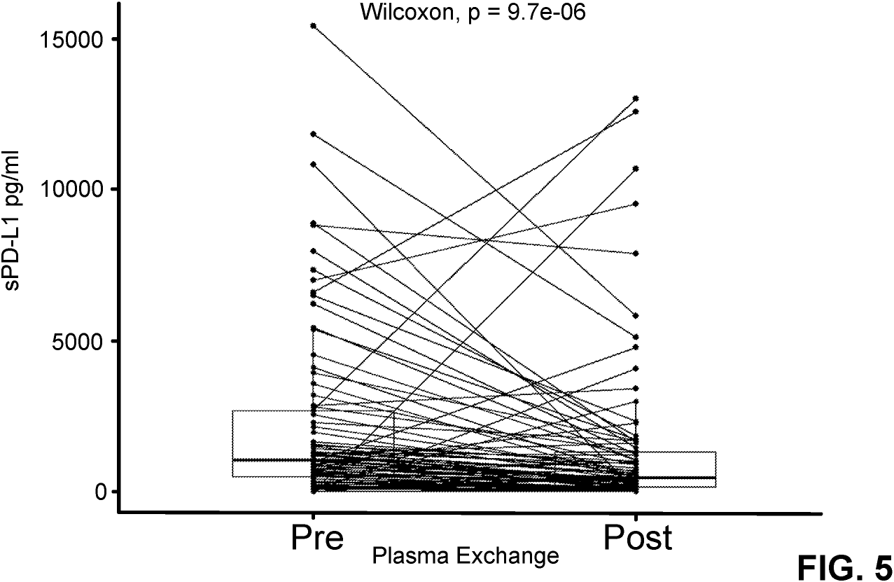

A representative individual patient treatment course showing sPD-L1 reduction over four successive TPE sessions is shown in FIG. 2C. All individual patient TPE courses, including sessions involving donated human blood products (e.g., FFP), are shown in FIG. 4. Pre- and post-TPE sPD-L1 levels for all sessions also are shown in FIG. 5. TPE significantly reduced plasma sPD-L1 even when sessions requiring donated FFP were included (p <0.0001).

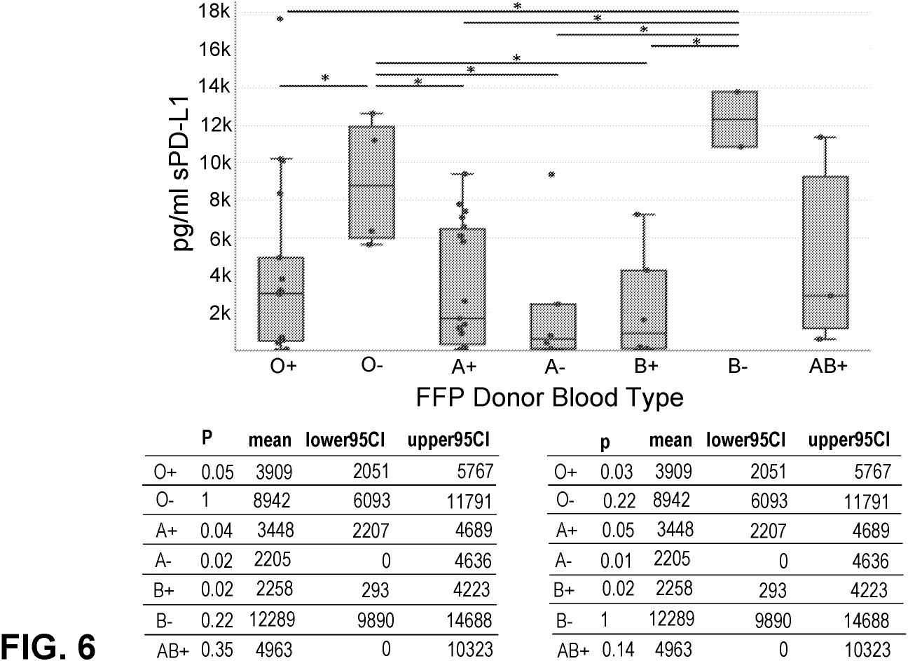

FFP is sometimes given during TPE for patients with increased risk of bleeding. It was observed that some patients receiving FFP with low baseline sPD-L1 experienced rapid increases in sPD-L1 levels after TPE, presumably passively acquired from donor plasma since this was not observed in patients receiving albumin replacement alone. sPD-L1 was not detected in the discarded plasma from the procedure for these patients. A mild association between post-FFP infusion rises in sPD-L1 levels and the blood type of the recipient was observed, mainly in patients with O type blood. Individuals with Group O-blood are universal recipients of FFP products and universal donors of cellular products due to a lack of ABO group antigens and the presence of pre-formed anti-A and anti-B antibodies, respectively. Recipients of FFP usually receive a mixture of compatible plasma from multiple donors. To determine whether blood type in FFP donors is associated with FFP sPD-L1 content, sPD-L1 was measured by ELISA in plasma from multiple FFP donors (FIG. 6). O-negative plasma donors showed higher sPD-L1 levels than donors with most other blood types.

### Example 4 - TPE Efficiently Reduces Plasma EV Levels in vivo

To determine whether TPE can remove PD-L1-positive EVs (evPD-L1) from patient blood, total EV levels and evPD-L1 were measured in each sample by flow cytometry. The impact of TPE on platelet-derived CD61-positive EVs and ADAM10-positive low density EVs also was determined as CD61-positive EVs are one of the most abundant EV subpopulations in blood (CD61 is a platelet marker), and ADAM10-positive low density EVs have been implicated in exosome loading and pathogenesis (Berckmans et al., Thromb Haemost, 85(4):639-646, 2001; Crescitelli et al., J Extracell Vesicles, 9(1):1722433, 2020; and Kowal et al., Proc Natl Acad Sci USA, 113(8):E968-E977, 2016).

TPE significantly reduced total plasma particle concentration (FIG. 7A, average 33.5% per exchange, p<0.0001). TPE sessions requiring FFP or other human blood product were excluded from analysis, leaving 44 session pairs. PD-L1-positive (evPD-L1) and ADAM10-positive EVs were significantly reduced by TPE (FIGS. 7B and 7C, p=0.028 and p<0.0001, respectively) and were detected in waste plasma (data not shown). Each TPE session using albumin-based replacement fluid with pre-TPE levels above one million removed a mean 73.1% of detectable PD-L1-positive EVs from patients (TABLE 6). Platelet-derived CD61-positive EVs, while abundant, were not significantly reduced by plasma exchange (FIG. 7D).

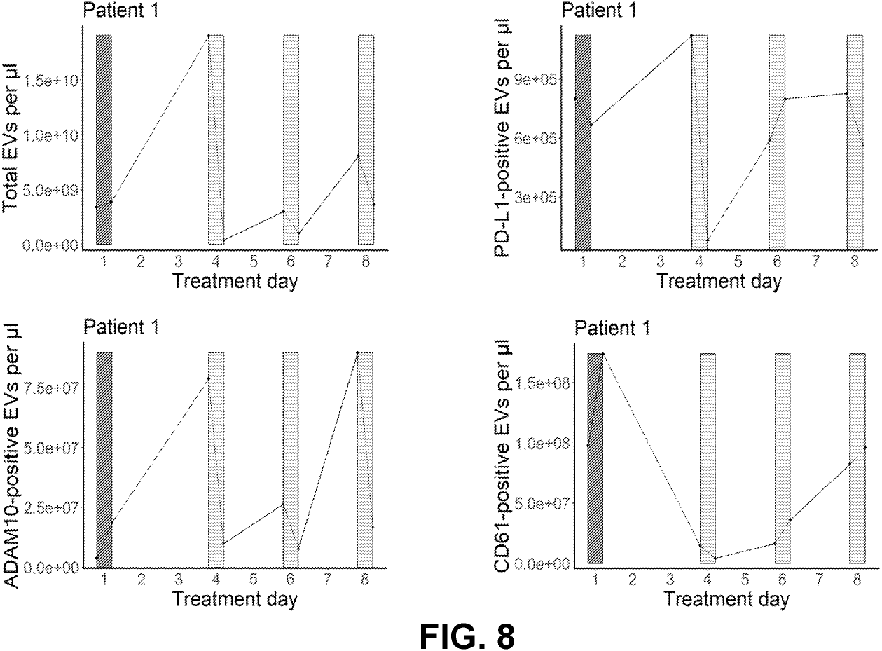

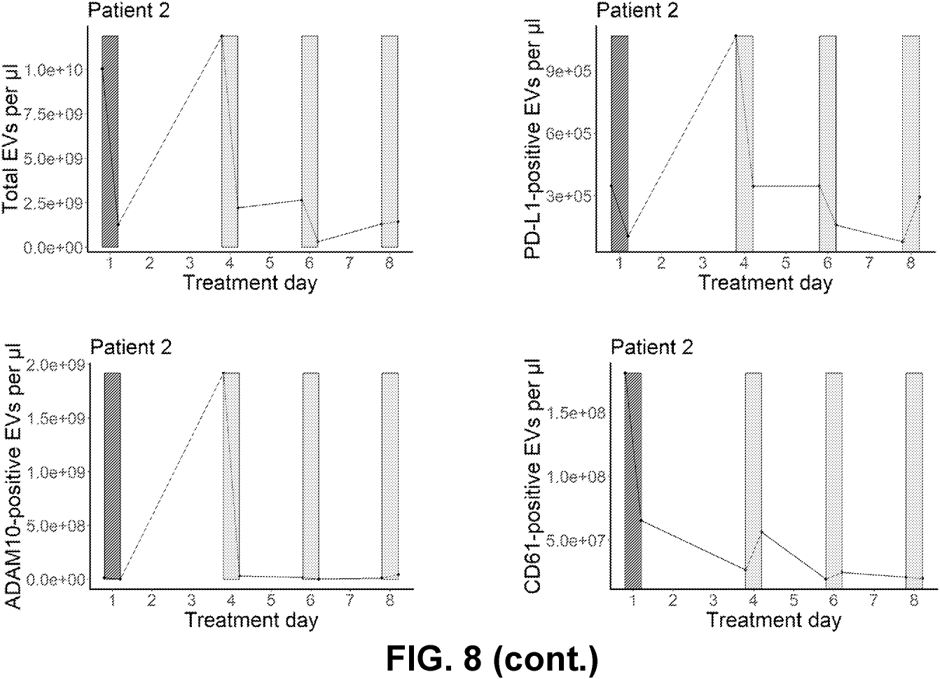

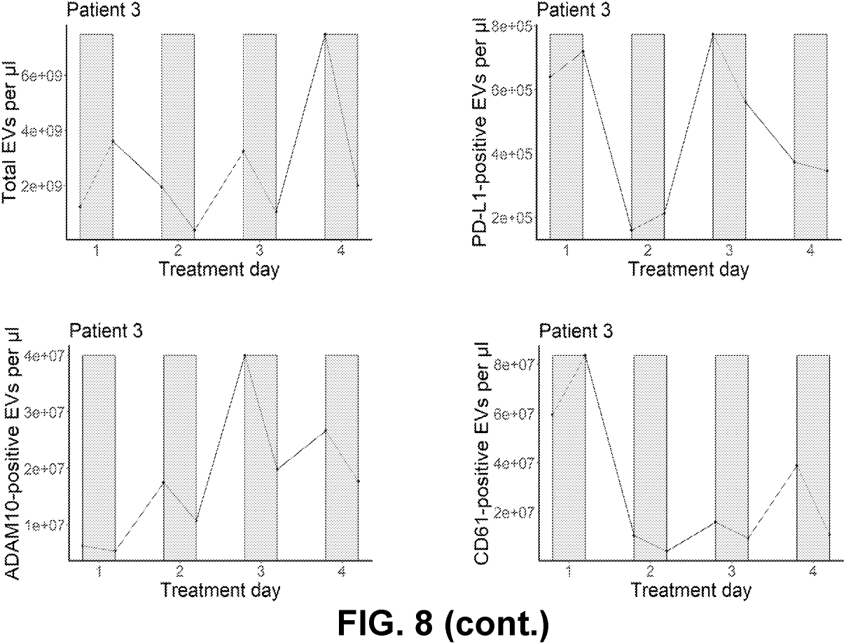

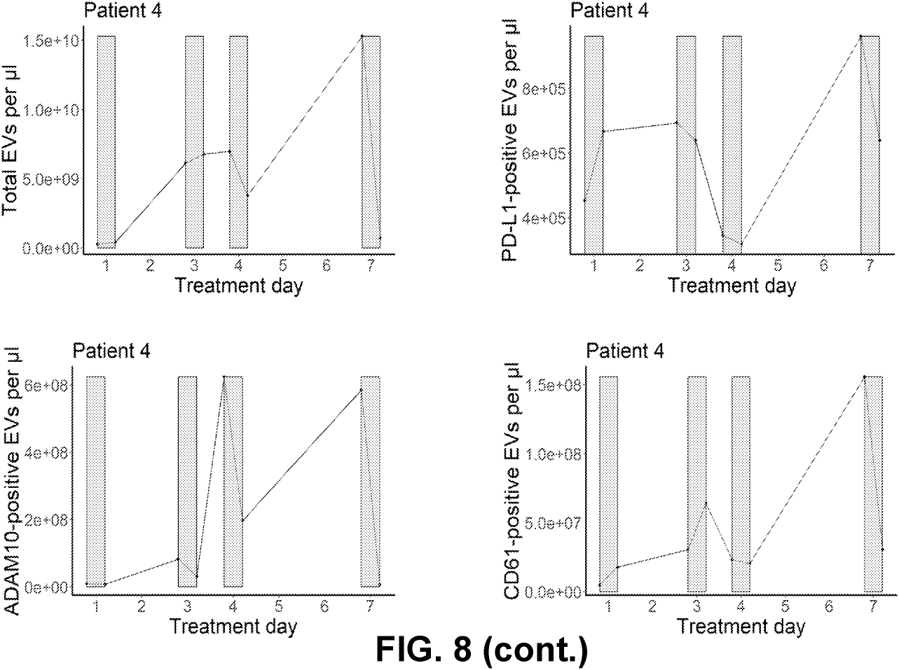

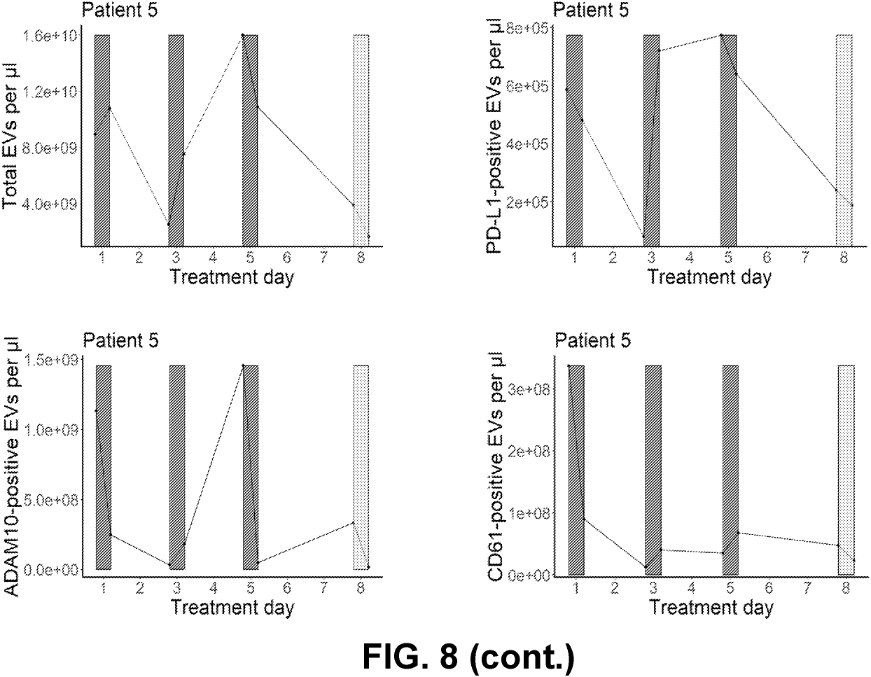

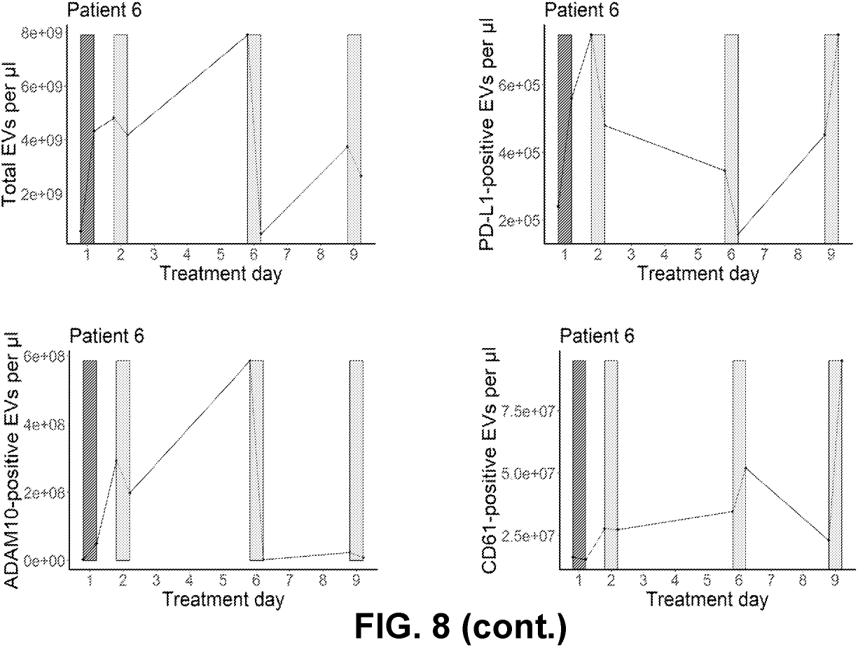

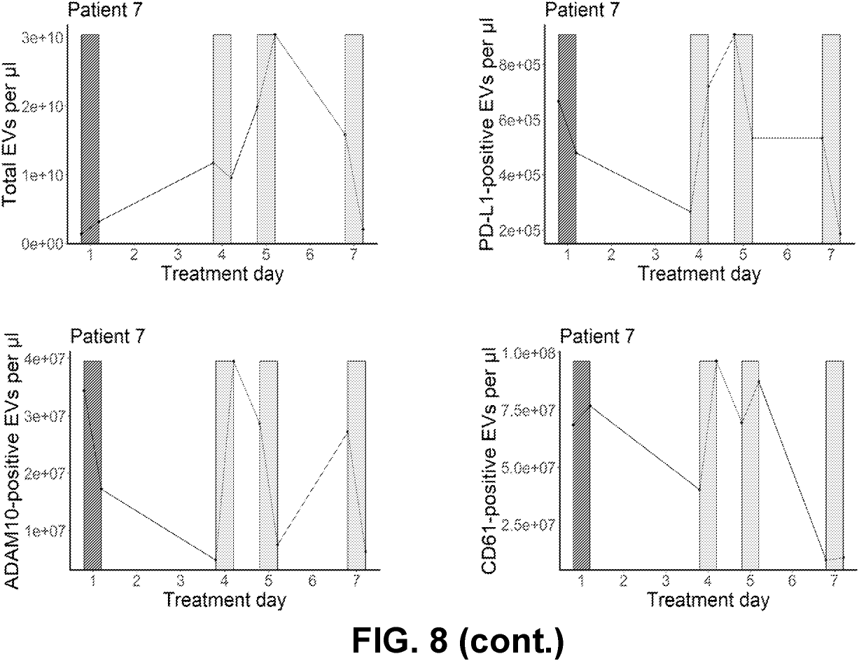

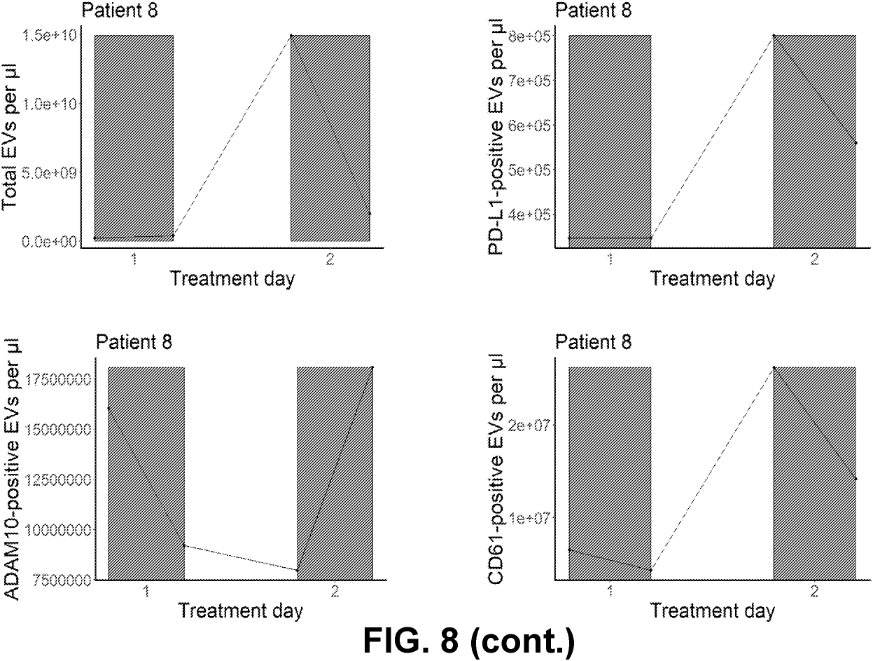

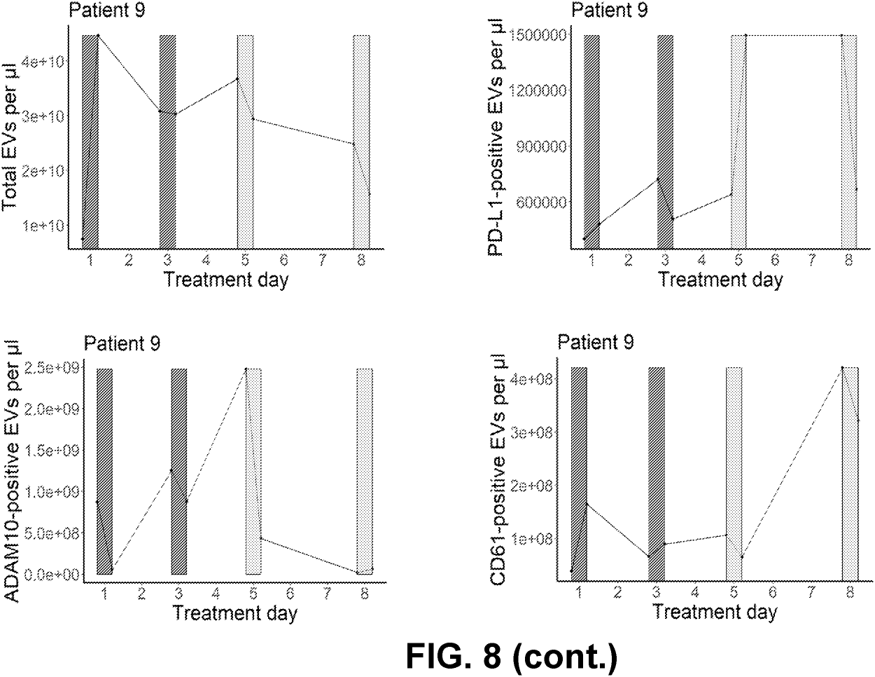

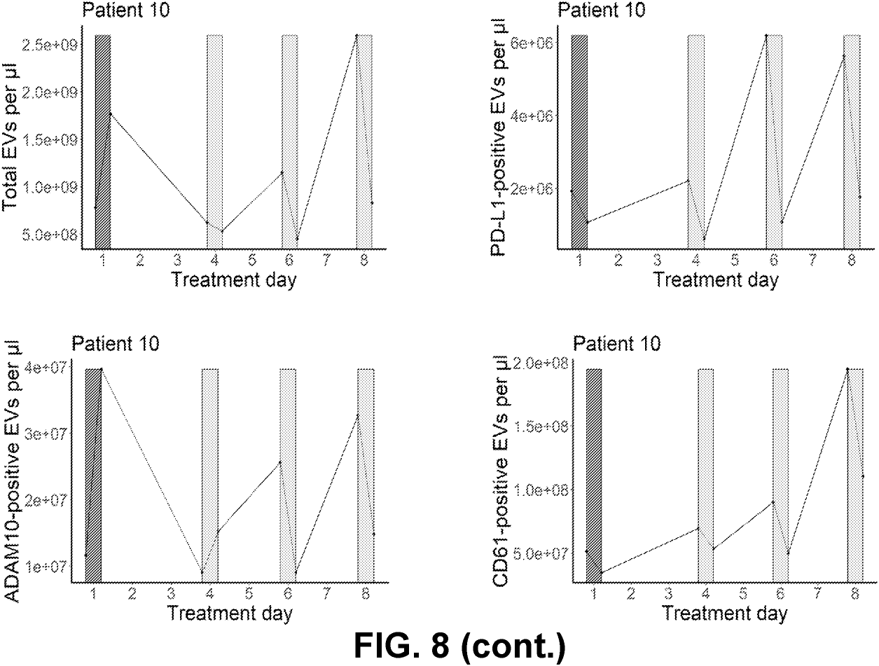

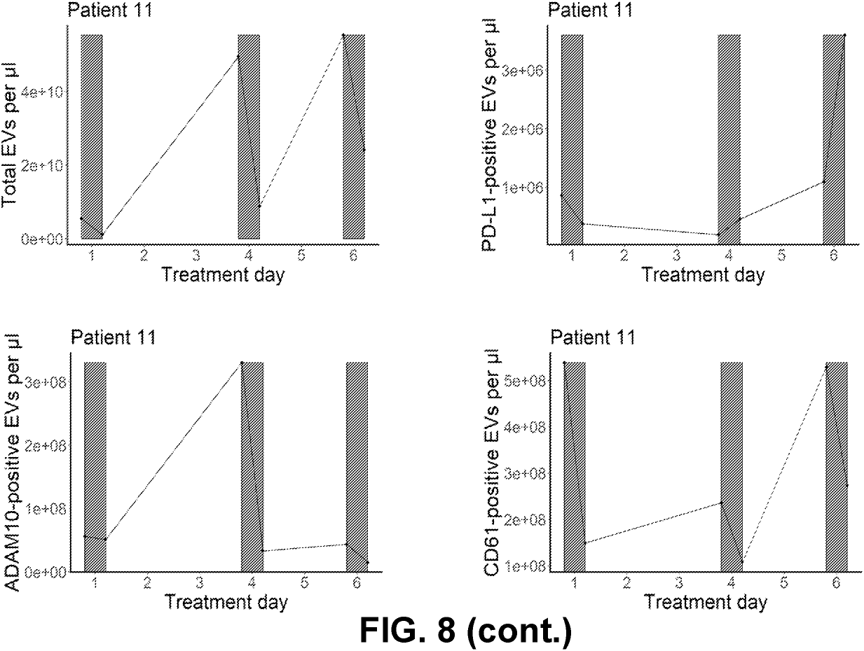

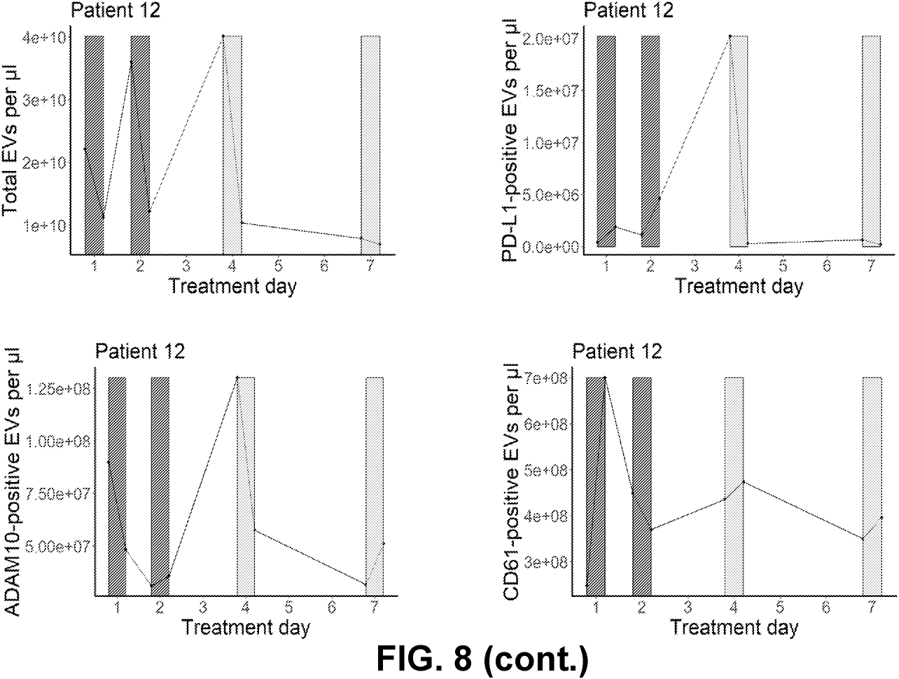

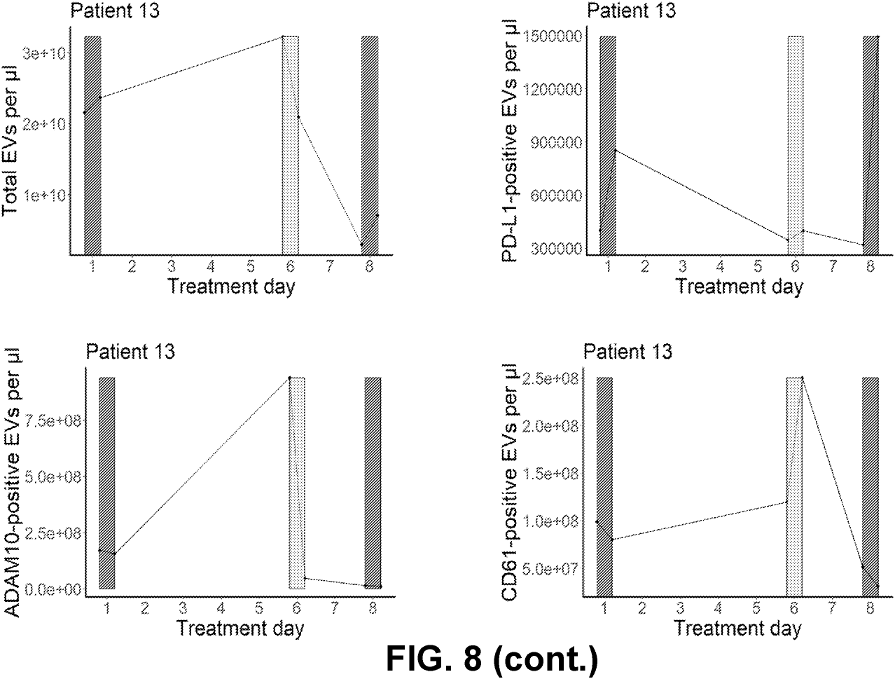

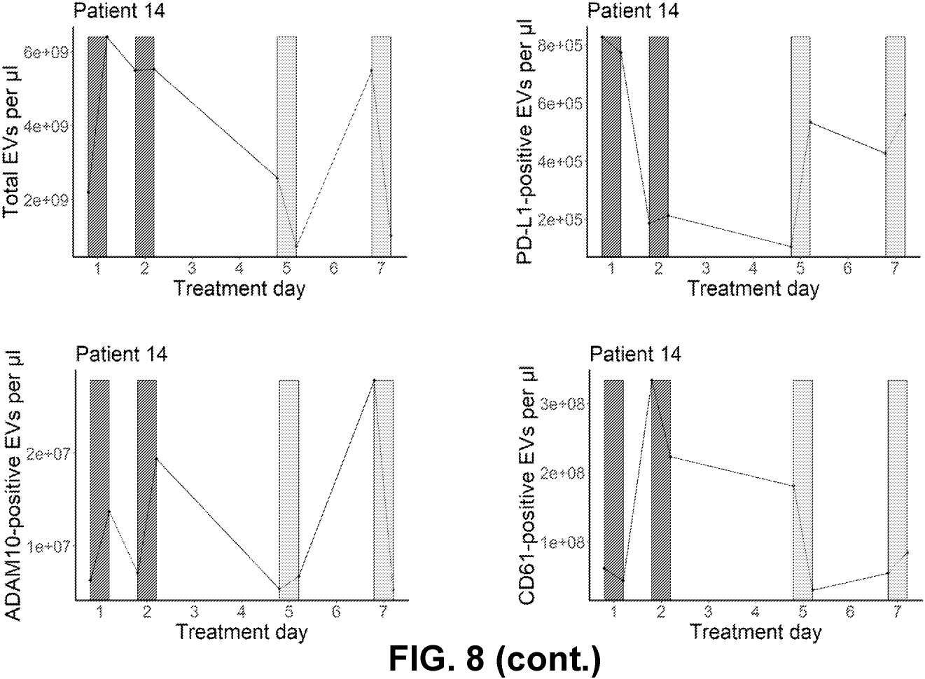

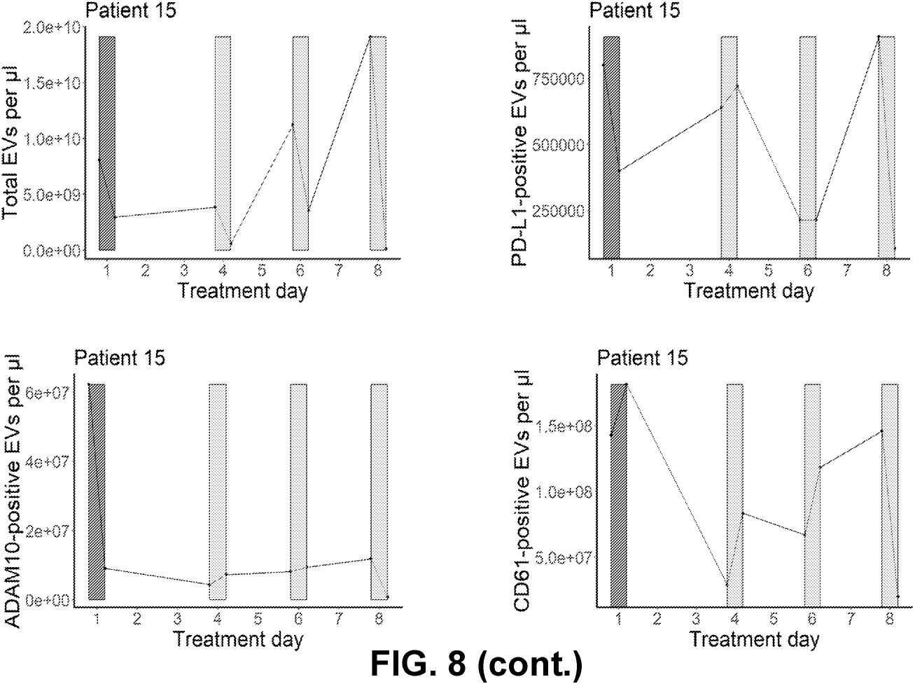

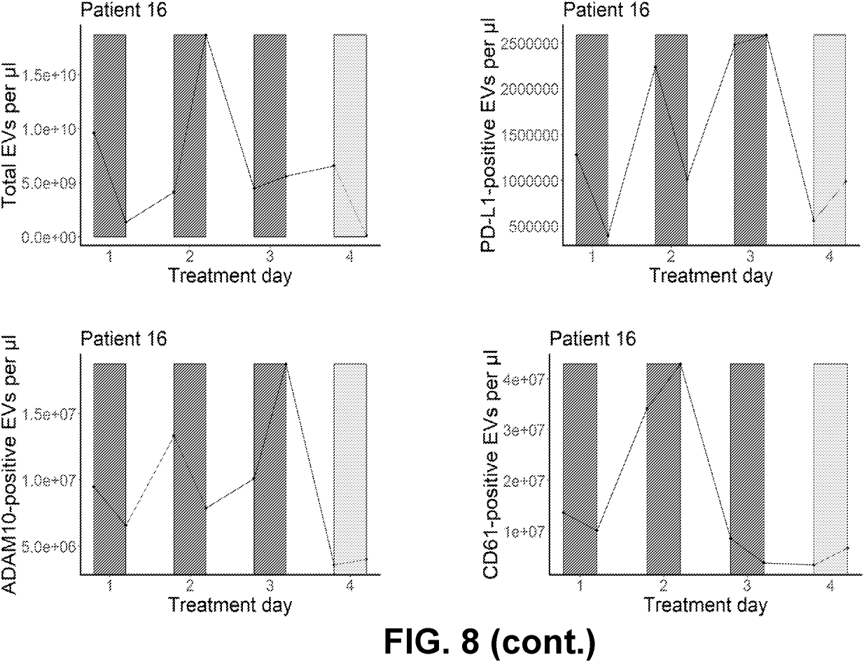

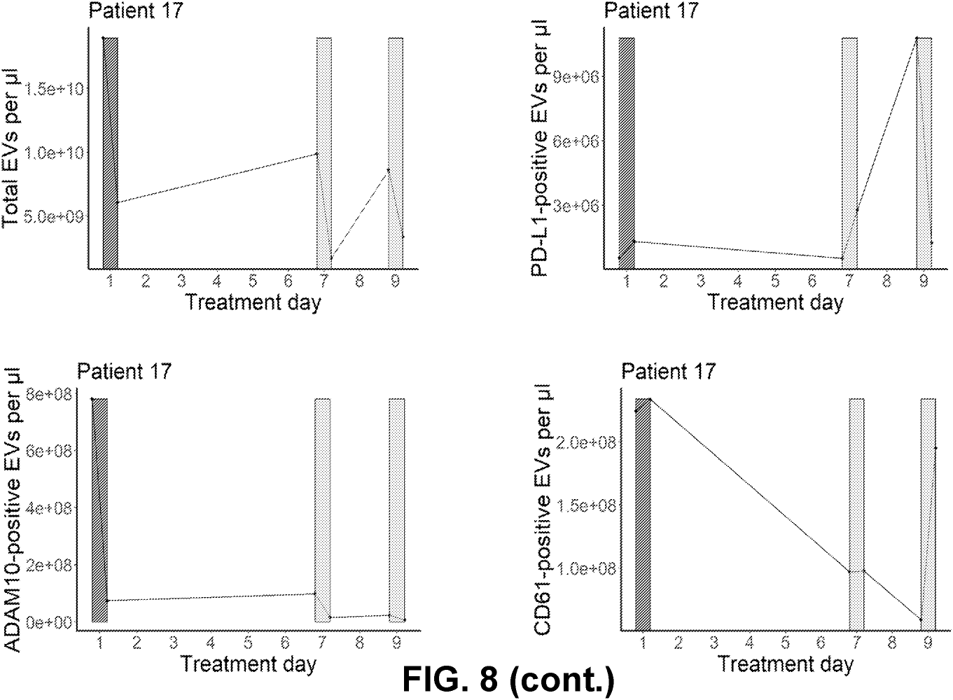

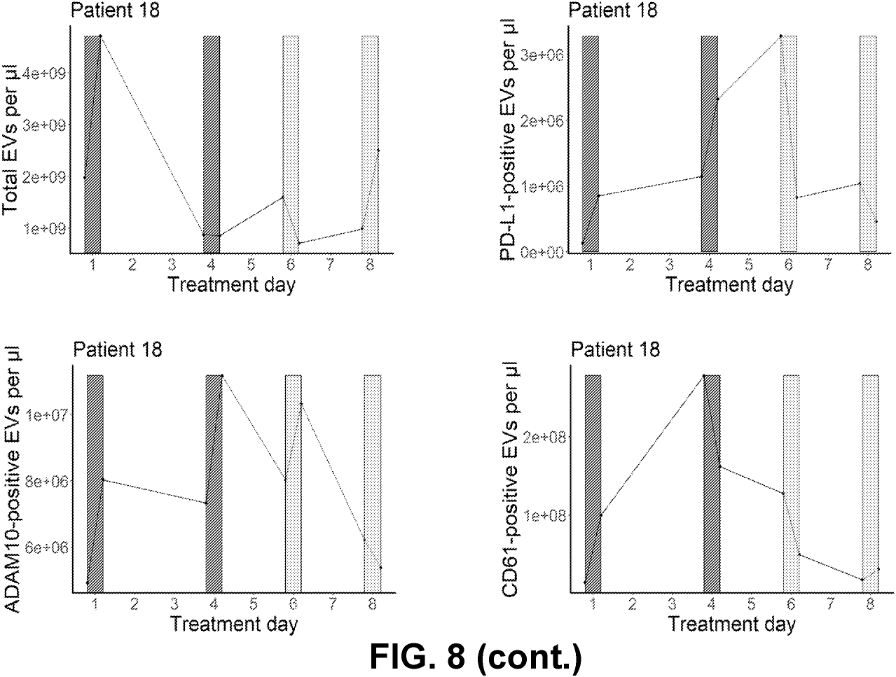

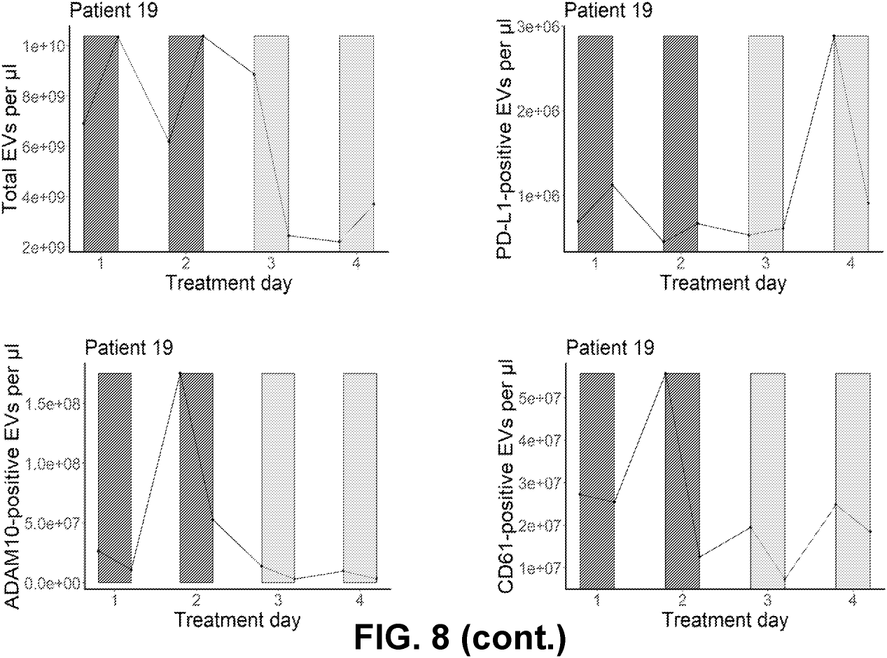

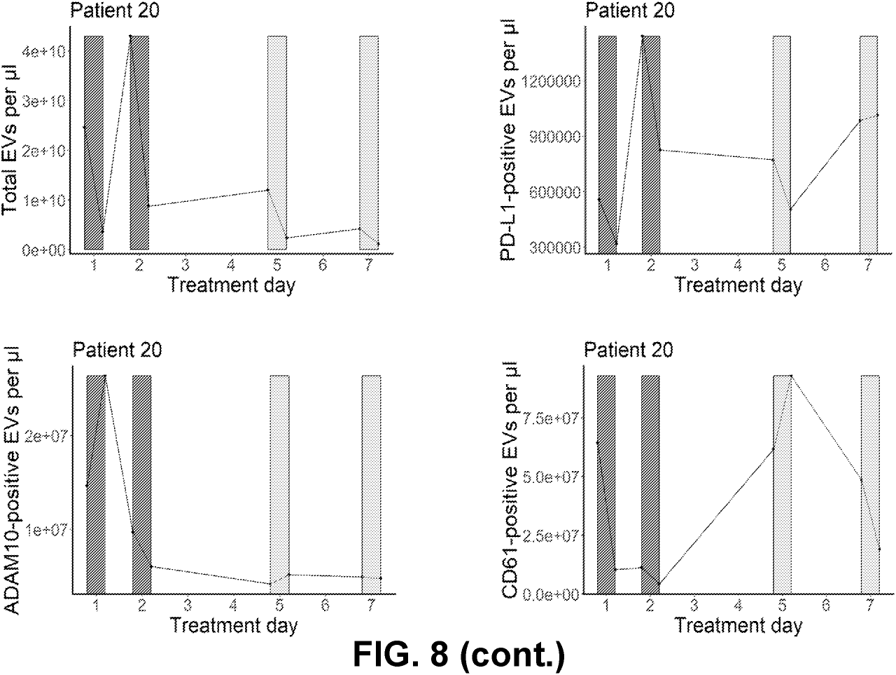

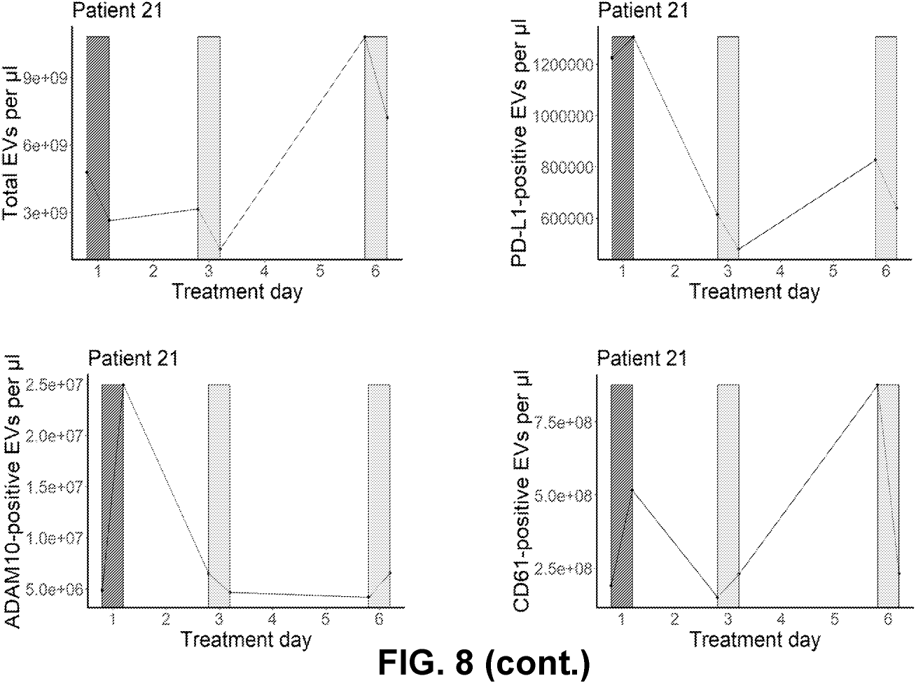

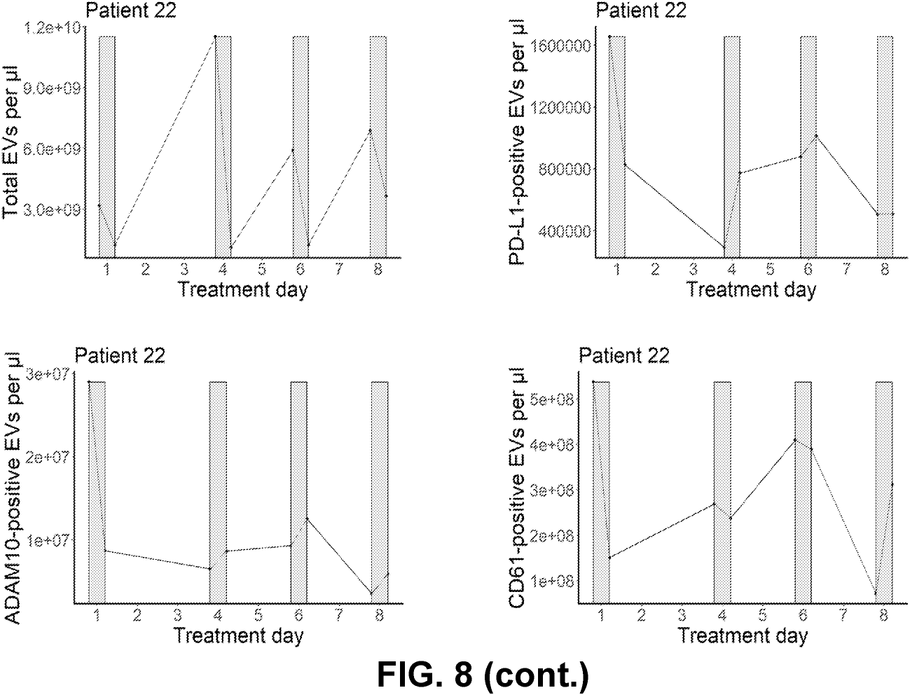

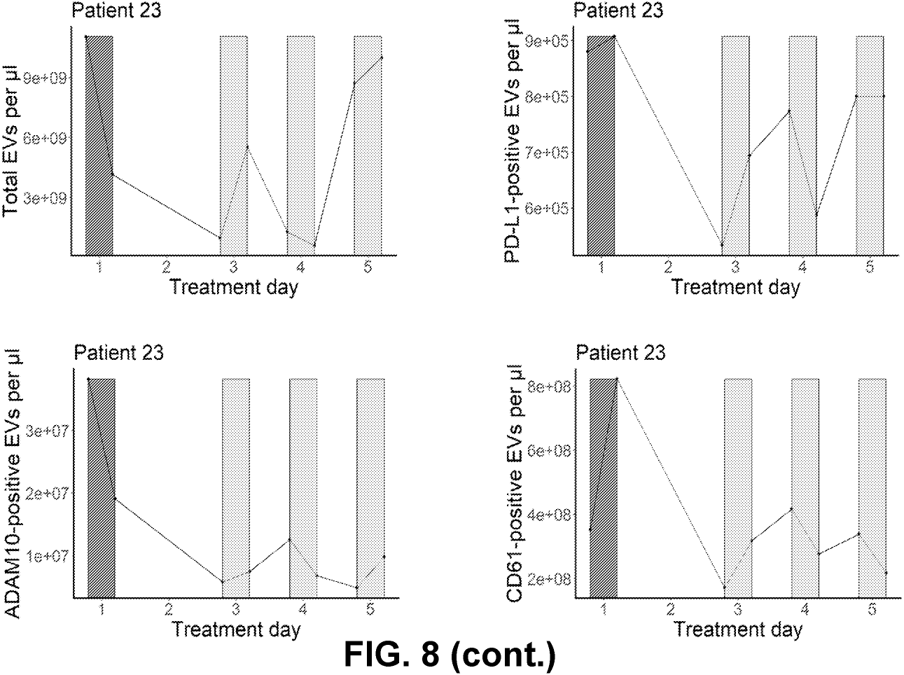

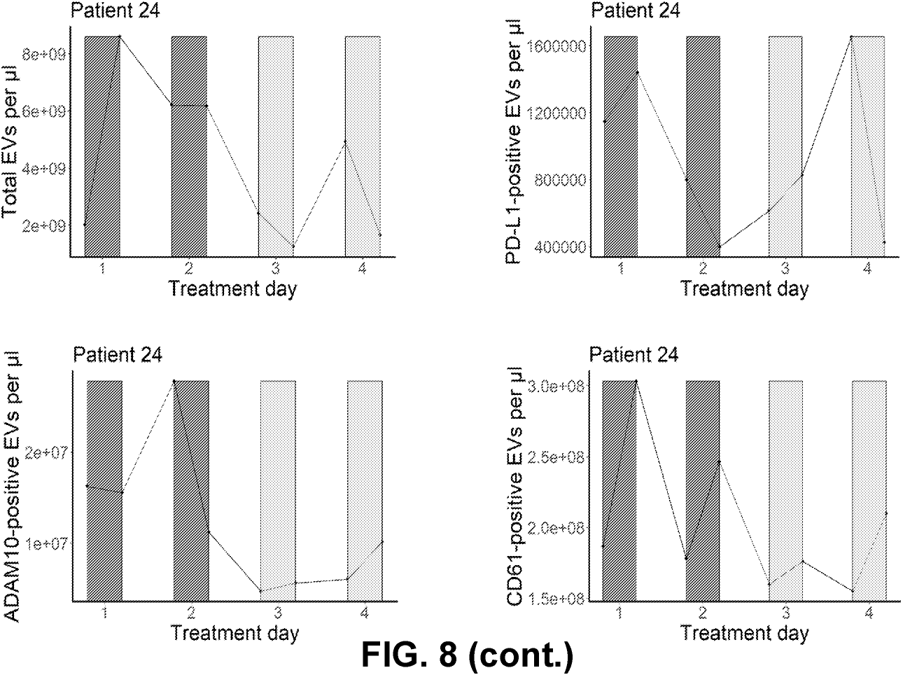

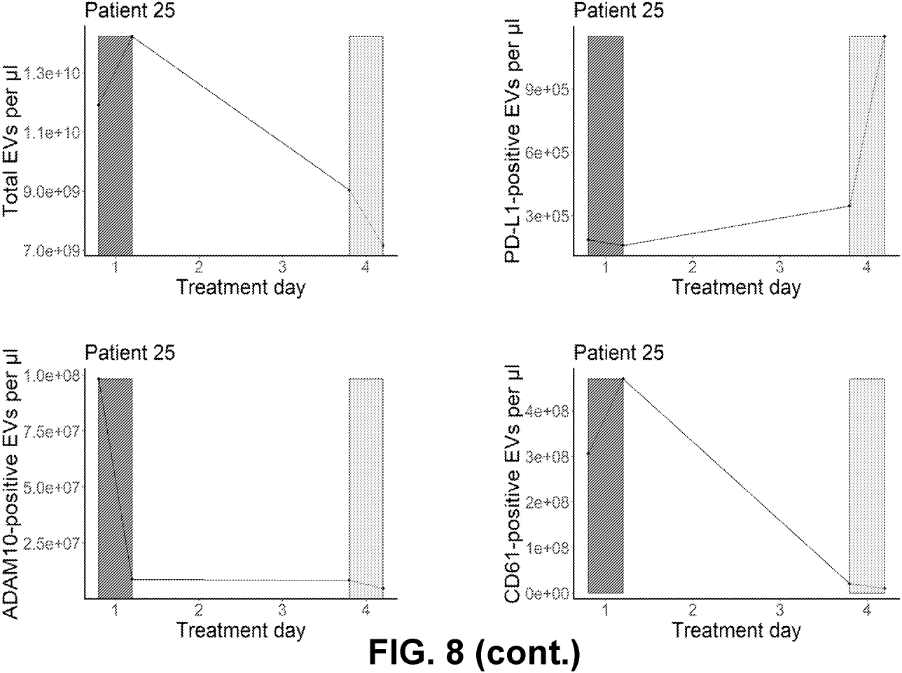

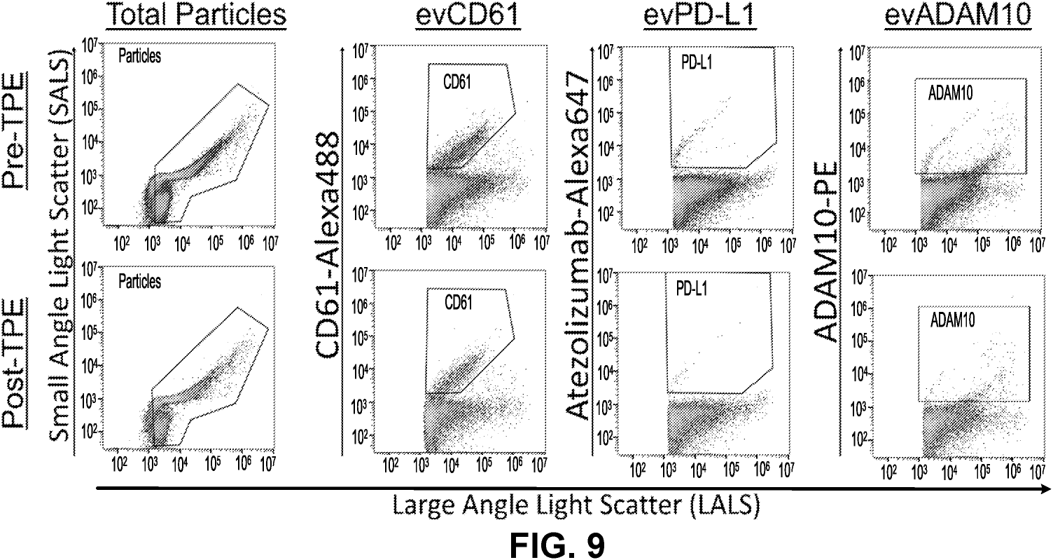

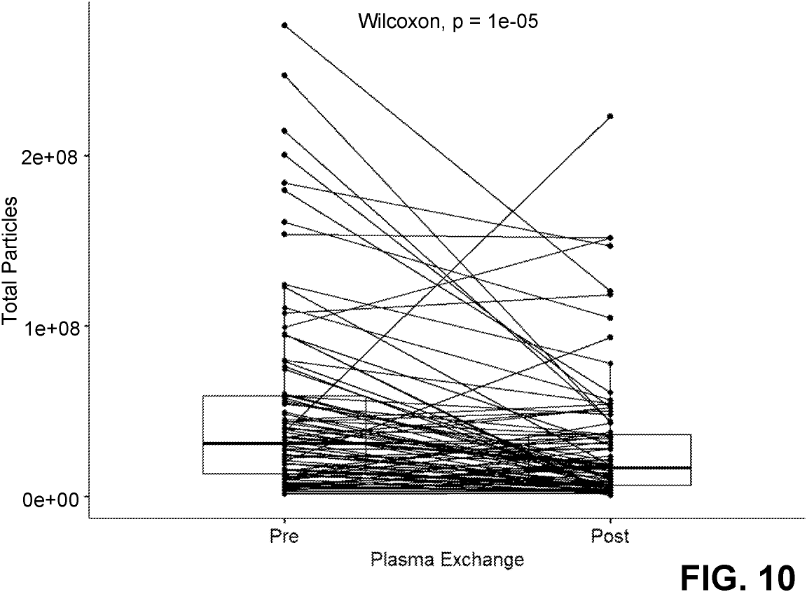

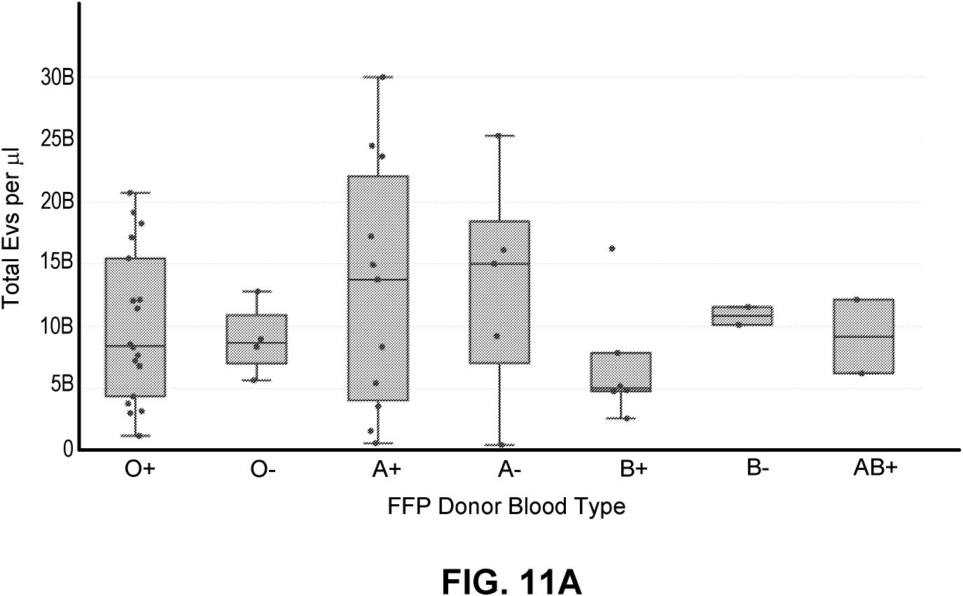

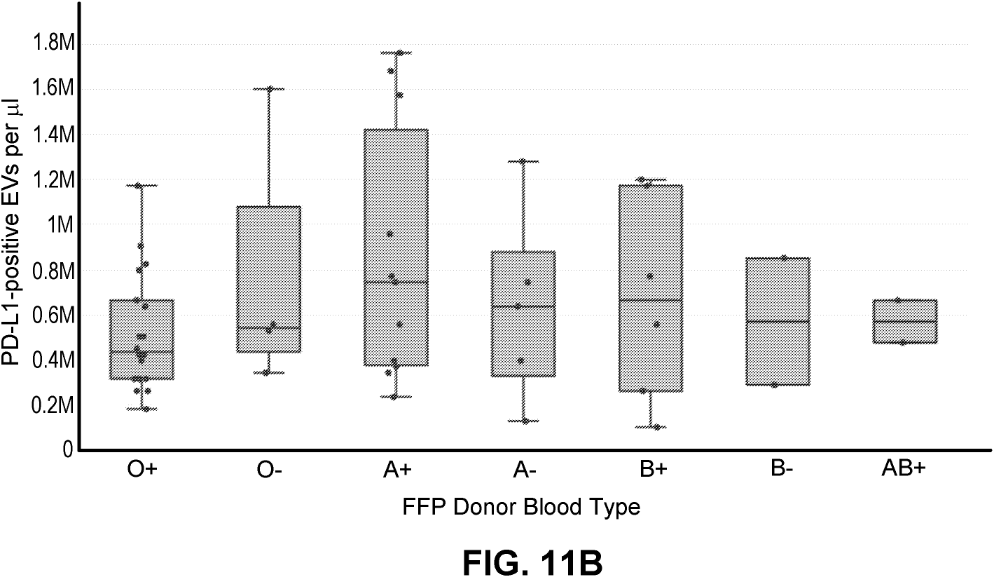

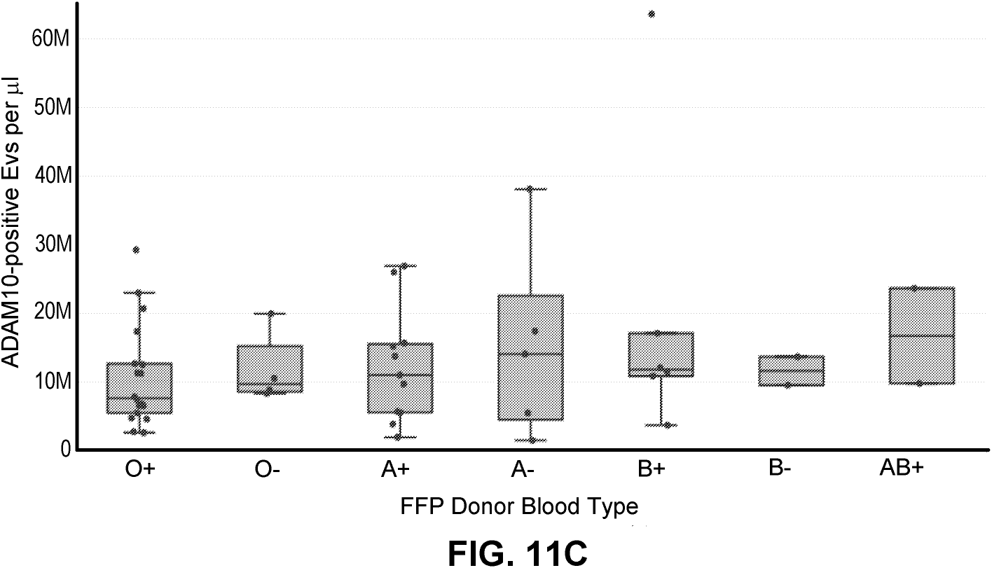

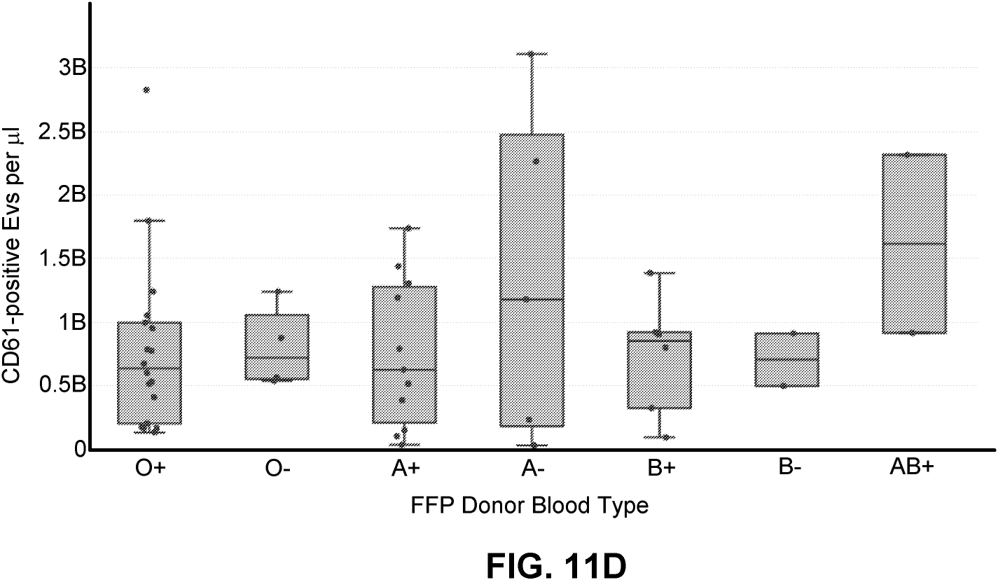

Individual patient courses showing total plasma, PD-L1-positive, ADAM10-positive, and CD61-positive EV levels before and after each TPE session are shown in FIG. 8, with exemplary nanoflow plots in FIG. 9. Three successive TPE sessions consistently depleted total, PD-L1-positive, and ADAM10-positive (but not CD61-positive) EVs. These trends were less pronounced when sessions in which patients received donor FFP were included (FIG. 10). In normal control FFP donors, blood type did not correlate with plasma EV concentrations (FIGS. 11A-11D). One patient in the study had melanoma and exhibited high pre-TPE evPD-L1 that was reduced by TPE. Another patient had a uterine neuroendocrine tumor and exhibited high pre-TPE sPD-L1 that was reduced by TPE. These patients’ tumors responded to PD-(L)1 treatment with pembrolizumab and atezolizumab, respectively.

TABLE 6

Reduction in EVs by subtype per exchange

| % Reduction in total EVs per exchange | (n=55) |
|---|---|
| Mean (SD) | 33.5 (89.4) |
| Median [Min, Max] | 60.9 [-468, 99.1] |
| % Reduction PD-L1-positive EVs per exchange | (n=13) |
| Mean (SD) | 73.1 (14.6) |
| Median [Min, Max] | 72.3 [50.0, 98.5] |
| % Reduction in ADAM10-positive EVs per exchange | (n=55) |
| Mean (SD) | 5.91 (126) |
| Median [Min, Max] | 42.4 [-709, 99.5] |

Percent reduction in EVs for TPE sessions in which over one million pre-TPE EVs were present (above background noise levels) and no FFP was given.

### Example 5 - TPE Significantly Reduces sPD-L1 Levels in Melanoma Patients Experiencing Progression After Immunotherapy

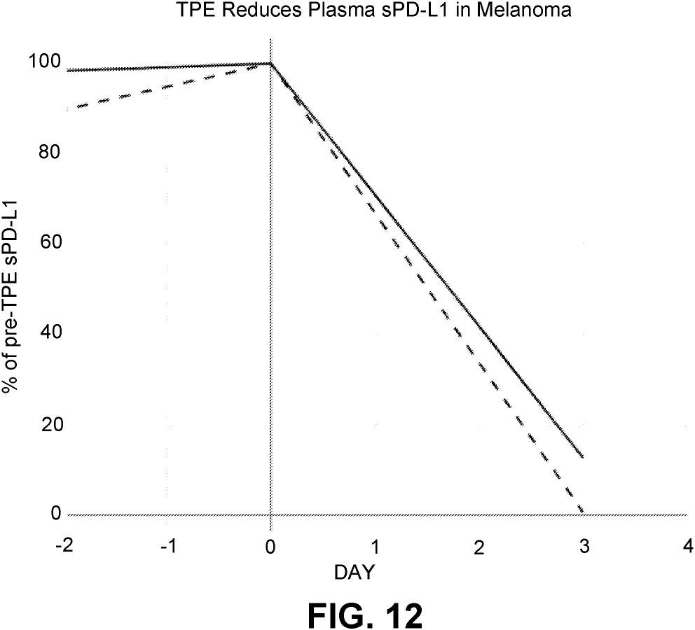

Patients with melanoma who had experienced disease progression despite immunotherapy with a PD-(L)1 inhibitor were screened for initial sPD-L1 levels in a prospective biomarker-selected trial. At screening, patients with sPD-L1 levels above a prespecified cutoff of 7.5 ng/mL were assessed for appropriate peripheral vascular access for the TPE procedure. Patients meeting the criteria underwent multiple days of TPE, and sPD-L1 levels were measured. Patients with high sPD-L1 levels at screening continued to show significant elevated sPD-L1 levels at the beginning of TPE after radiation. After TPE, all patients in the study experienced significant decreases in sPD-L1 levels (FIG. 12).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
(a) performing therapeutic plasma exchange (TPE) on a mammal identified as (i) having cancer and (ii) having a measured level of one or more markers in a biological sample that is equal to or higher than a threshold level, and subsequently
(b) administering an immunotherapy to the mammal.

2. The method of claim 1, wherein said one or more markers comprises soluble PD-L1 (sPD-L1) or extracellular vesicle PD-L1 (evPD-L1).

3. The method of claim 2, wherein said one or more markers comprises sPD-L1, and wherein said threshold level is from 0.27 ng/mL to 1.75 ng/mL sPDL1.

4. The method of claim 2, wherein said one or more markers comprises sPD-L1, and wherein said threshold level is from 5 ng/mL to 10 ng/mL sPDL1.

5. (canceled)

6. The method of claim 1, wherein said one or more markers comprises extracellular vesicles (EVs).

7. The method of claim 1, wherein said measuring comprises performing an enzyme linked immunosorbent assay (ELISA) or performing nanoflow cytometry.

8. (canceled)

9. The method of claim 1, wherein said immunotherapy comprises an anti-PD-1 antibody or anti-PD-L1 antibody.

10. The method of claim 1, wherein said mammal is a human.

11. The method of claim 1, wherein said cancer is melanoma, renal cell carcinoma, mesothelioma, squamous cell cancer, a hematological cancer, neurological cancer, breast cancer, head and neck cancer, gastrointestinal cancer, liver cancer, pancreatic cancer, genitourinary cancer, bone cancer, bladder cancer, or vascular cancer.

12. A method for treating a mammal identified as having cancer, said method comprising:
(a) measuring the level of one or more markers in a biological sample obtained from said mammal,
(b) comparing said measured level of said one or more markers to a threshold level,
(c) when the measured level is equal to or greater than said threshold level, performing TPE on the mammal, and subsequently
(d) administering an immunotherapy to the mammal.

13. The method of claim 12, wherein said one or more markers comprises sPD-L1 or evPD-L1.

14. The method of claim 13, wherein said one or more markers comprises sPD-L1, and wherein said threshold level is from 0.27 ng/mL to 1.75 ng/mL sPDL1.

15. The method of claim 13, wherein said one or more markers comprises sPD-L1, and wherein said threshold level is from 5 ng/mL to 10 ng/mL sPDL1.

16. (canceled)

17. The method of claim 12, wherein said one or more markers comprises EVs.

18. The method of claim 12, wherein said measuring comprises performing an ELISA or performing nanoflow cytometry.

19. (canceled)

20. The method of claim 12, wherein said immunotherapy comprises an anti-PD-1 or anti-PD-L1 antibody.

21. The method of claim 12, wherein said mammal is a human.

22. The method of claim 12, wherein said cancer is melanoma, renal cell carcinoma, mesothelioma, squamous cell cancer, a hematological cancer, neurological cancer, breast cancer, head and neck cancer, gastrointestinal cancer, liver cancer, pancreatic cancer, genitourinary cancer, bone cancer, bladder cancer, or vascular cancer.

23. A method for treating a mammal identified as having a cancer that is resistant to immunotherapy, wherein the method comprises performing TPE on said mammal and administering an immunotherapy to said mammal.

24. The method of claim 23, wherein said mammal was identified as having a cancer resistant to immunotherapy by measuring the level of one or more immunosuppressive components in a biological sample obtained from said mammal, and determining that said level is equal to or higher than a given cutoff level.

25. The method of claim 23, wherein said one or more immunosuppressive components comprise one or more of sPD-L1, evPD-L1, and evADAM10.

26. The method of claim 23, wherein said mammal is a human.


SEQUENCE LISTING (1 sequence)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET